(19) United States
(12) Patent Application Publication
(10) Pub. No.: US 2026/0311830 A1
(43) Pub. Date: Oct. 8, 2026

(54) MACHINE-LEARNING ARCHITECTURES FOR INTERACTION DATA GENERATION

(71) Applicant: MYRIAD SYSTEMS INC., Valhalla, NY (US)

(72) Inventors: Jeremy SHINER, Valhalla, NY (US); Enis TAHIRAJ, Valhalla, NY (US)

(73) Assignee: MYRIAD SYSTEMS INC., Valhalla, NY (US)

(21) Appl. No.: 19/637,877

(22) Filed: Apr. 2, 2026

Related U.S. Application Data
(60) Provisional application No. 63/783,132, filed on Apr. 3, 2025.

(51) Int. Cl. G16H 15/00; G06F 3/04815; G06F 40/174; G06T 17/00; G06T 19/00; G10L 15/26

(52) U.S. Cl. CPC G16H 15/00; G06F 3/04815; G06F 40/174; G06T 17/00; G06T 19/00; G06T 2200/24; G06T 2210/41; G06T 2219/004; G10L 15/26

(57) ABSTRACT

Systems and methods of handling or generating patient-related data across a variety of data sources and modalities use various artificial intelligence models. A computer receives patient-supplied data and data from trusted sources to detect discrepancies. The computer can generate template forms for obtaining and analyzing patient-supplied data. A computer generates a 3D model user interface using patient-related data. A machine-learning model converts patient data into 3D representations of the patient anatomy. The computer uses an LLM to generate clinical notes, which the computer may use as directly on the 3D model.

### CROSS-REFERENCE TO RELATED APPLICATION

This application claims the benefit of and priority to US Provisional Application No. 63/783,132, titled “Machine-Learning Architectures for Clinical Documentation Ingestion and Generation,” filed Apr. 3, 2025, which is incorporated by reference in its entirety.

### TECHNICAL FIELD

The present disclosure generally relates to systems and methods for automated clinical documentation. More specifically, embodiments include machine-learning architectures to improve the accuracy and efficiency of capturing and generating clinical documentation for patients for healthcare providers.

### BACKGROUND

Existing technologies for capturing doctor notes typically involve manual entry methods, voice recognition software, and template-based systems. Manual entry methods require healthcare providers to type or write notes for patient care, consuming valuable time and increasing the risk of errors. Template-based systems provide structured formats for data entry to create patient notes, but such conventional software often lacks the flexibility to accommodate varying clinical scenarios, leading to incomplete or inaccurate documentation and/or time consuming manual documentation. Template-based systems, while providing some structure, are often too rigid to capture the nuances of individual patient encounters. There are further technological shortcomings in these existing methods, including inefficiencies in time management, potential for errors, and lack of adaptability to diverse clinical environments. Manual entry methods are labor-intensive and prone to human error.

Another common approach to create patient-care notes involves voice recognition software for converting healthcare provider's spoken words into text, allowing healthcare providers to dictate patient notes for patient care. However, accuracy of voice recognition remains a significant challenge in recognizing and capturing spoken content, particularly with respect to unusual or complicated medical terminology. Typical voice recognition software struggles with the precise interpretation of complex medical terms or handling spoken accents, resulting in frequent inaccuracies. Such systems often miss inputs from patients and lack the ability add supporting content.

Desired technological solutions involve systems capable of accurately and efficiently capturing comprehensive clinical documentation. Improved accuracy in transcription and interpretation of medical terminology, adaptability to various clinical settings, and seamless integration with existing electronic health record systems are essential. These advancements would address current inefficiencies, reduce the potential for errors, and ensure complete and accurate documentation of patient encounters.

Another significant shortcoming in technologies for clinical documentation relates to the technological integration of disparate systems used for capturing doctor's notes. Many current solutions are not well-integrated with electronic health record systems (EHRs), leading to a disconnected workflow that hampers efficient data management. This lack of interoperability often necessitates redundant data entry, increases administrative workload, and can result in discrepancies within patient records. Moreover, the absence of a unified platform capable of harmonizing various data sources such as lab results, imaging, and patient histories complicates the task of maintaining comprehensive and up-to-date clinical documentation. These integration issues highlight the critical need for advanced systems that can seamlessly interact with EHRs and/or separate and disjointed practice management systems, ensuring accurate, efficient, and cohesive data management across different healthcare platforms for capturing patient notes and clinical documentation.

### SUMMARY

Disclosed herein are systems and methods capable of addressing the above-described shortcomings and may also provide any number of additional or alternative benefits and advantages. Embodiments include computing systems of an automated clinical documentation system that implements various types of machine-learning architectures to generate patient narratives using various forms of Artificial Intelligence (AI) and machine-learning techniques, including generative AI models. The computing system captures various types of patient data across any number of systems or modalities and one or more machine-learning architectures, including generative machine-learning models, generative neural networks, (or generative artificial intelligence models), such as a Large Language Model (LLM), for ingesting, integrating, and interpreting the disparate forms of data to output AI-generated narratives (sometimes referred to as clinical notes or clinical interaction data) and other forms of downstream functions.

Embodiments may include computer-implemented method for generating subject-related information using machine-learning architectures of a data system, the method including: obtaining, by a computer, subject-related data for a subject associated with a subject interaction instance from a plurality of data sources; generating, by the computer, interaction data for the interaction instance using a generative machine-learning model based upon the subject-related data; generating, by the computer, a 3D model image representing a morphology of the subject based on the subject-related data; and generating, by the computer, an interactive 3D model user interface that includes the 3D model and an overlay of an exposition containing the interaction data associated with the morphology of the subject for display at a service computing device.

The may method may further include receiving, by the computer, a service-supplied input from a service computing device interacting with the interactive 3D model user interface.

The may method may further include updating, by the computer, a portion of the subject-related data for the subject based upon the service-supplied input entered to the interactive 3D model user interface.

The method may further include updating, by the computer, the 3D model and the exposition based on the subject-related data; and updating, by the computer, the interactive 3D model user interface according to the 3D model and the exposition as updated for the service-supplied input.

When obtaining the subject-related data, the method may further include: obtaining, by the computer, an audio signal data containing a speech signal for one or more speakers including a service; and generating, by the computer, a portion of the subject-related data based upon converting at least a portion of the speech signal of the service to text data for the portion of the subject-related data.

The computer may receive the audio signal data via a microphone having the speech signal for the service and the subject during the interaction instance. The computer may generate the interaction data using the subject-related data having the text data converted using the speech signal during the interaction instance.

When generating the interaction data for the interaction instance using the generative machine-learning model, the method may further include obtaining, by the computer, a generative machine-learning model prompt stored in a database and containing instructs for the generative machine-learning model associated with the service for generating the interaction data.

When obtaining the subject-related data, the method may further include extracting, by the computer, a first feature vector for subject-supplied input data using the subject-related data, and a second feature vector for trusted-source input data using the subject-related data; and generating, by the computer, a discrepancy score for the subject-supplied input data based upon a distance between the first feature vector and the second feature vector.

The method may further include detecting, by the computer, a discrepancy in the subject-related data in response to determining that the discrepancy score satisfies a detecting threshold; and generating, by the computer, an alert indicating the discrepancy for display at a user interface.

When obtaining the subject-related data for the subject, the method may further include: obtaining, by the computer, a subject-intake form from a database containing a plurality of templated forms; and generating, by the computer, a user interface for displaying the subject-intake form at a subject device. The computer receives at least a portion of the subject-related data as subject-supplied data inputs entered at the user interface having the subject-intake form.

Embodiment may include a system for generating subject-related information using machine-learning architectures. The system may include a server having at least one processor, configured to: obtain subject-related data for a subject associated with a interaction instance from a plurality of data sources; generate interaction data for the interaction instance using a generative machine-learning model based upon the subject-related data; generate a 3D model image representing a morphology of the subject based on the subject-related data; and generate an interactive 3D model user interface that includes the 3D model and an overlay of an exposition containing the interaction data associated with the morphology for display at a service computing device.

The server may be further configured to receive a service-supplied input from a service computing device interacting with the interactive 3D model user interface.

The server may be further configured to update a portion of the subject-related data for the subject based upon the service-supplied input entered to the interactive 3D model user interface.

The server may be further configured to: update the 3D model and the exposition based on the subject-related data; and update the interactive 3D model user interface according to the 3D model and the exposition as updated for the service-supplied input.

When obtaining the subject-related data, the server may be configured to: obtain an audio signal data containing a speech signal for one or more speakers including a service; and generate a portion of the subject-related data based upon converting at least a portion of the speech signal of the service to text data for the portion of the subject-related data.

The server may receive the audio signal data via a microphone having the speech signal for the service and the subject during the interaction instance. The server may generate the interaction data using the subject-related data having the text data converted using the speech signal during the interaction instance.

When generating the interaction data for the interaction instance using the generative machine-learning model, the server may be configured to obtain a generative machine-learning model prompt stored in a database and containing instructs for the generative machine-learning model associated with the service for generating the interaction data.

When obtaining the subject-related data, the server may be configured to: extract a first feature vector for subject-supplied input data using the subject-related data, and a second feature vector for trusted-source input data using the subject-related data; and generate a discrepancy score for the subject-supplied input data based upon a distance between the first feature vector and the second feature vector.

The server may be further configured to: detect a discrepancy in the subject-related data in response to determining that the discrepancy score satisfies a detecting threshold; and generate an alert indicating the discrepancy for display at a user interface.

When obtaining the subject-related data for the subject, the server may be further configured to: obtain a subject-intake form from a database containing a plurality of templated forms; and generate a user interface for displaying the subject-intake form at a subject device. The server receives at least a portion of the subject-related data as subject-supplied data inputs entered at the user interface having the subject-intake form.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1A depicts the various device components of the system in a networked environment, according to some embodiments.

FIG. 1B depicts an example collection of executable software engines of analytics servers, according to some embodiments.

FIG. 2 shows a flowchart of operations of a computer-implemented method for implementing a LLM for generating textual clinical notes for a patient using any number of inputs containing patient-related data, according to some embodiments.

FIG. 3 shows a flowchart of operations of a computer-implemented method for implementing machine-learning models of a machine-learning architecture for detecting discrepancies in subject-supplied (or patient-supplied) data inputs, as compared against provider-supplied data or stored patient data.

FIG. 4 shows a flowchart of operations of a computer-implemented method for generating 3D anatomy models of patient anatomies for a 3D model interface, according to some embodiments.

FIG. 5 shows a flowchart of operations of a computer-implemented method for generating a text transcription or other form of output using audio signal data containing speech signals from one or more speakers (e.g., provider, patient), according to some embodiments.

FIG. 6 is a graphical user interface displaying an example of “mapped” data, using keywords and machine-learning model for a subsection of the clinical note.

FIG. 7 is a graphical user interface displaying a “chat style” response with an LLM.

### DETAILED DESCRIPTION

Reference will now be made to the illustrative embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to a person skilled in the relevant art and having possession of this disclosure, are to be considered within the scope of the invention.

Embodiments may perform operations for automatic narrative generation and diagnostic code predictions or suggestions. A computer may execute programming for a machine-learning architecture implementing a machine-learning model, such as Natural Language Processing (NLP), a Large Language Model (LLM), or other generative machine-learning model, trained for automatic narrative generation to generate clinical notes based on various types of data inputs and data sources. The same or different machine-learning model may be further trained to suggest relevant diagnosis codes, such as Current Procedural Terminology (CPT) codes, based on the content of the clinical note or patient data.

A generative machine-learning model may include a machine-learning model configured to generate output data (e.g., sequences of tokens and/or strings of text) conditioned on input data. In healthcare implementations, a generative machine-learning model may generate interaction data such as clinical notes, summaries, sections (e.g., SOAP sections), recommendations, and/or other narratives for a patient visit/interaction instance. Non-limiting examples of generative machine-learning models include transformer-based language models (including LLMs), encoder-decoder sequence-to-sequence models, and other neural network architectures configured for conditional text generation based on prompts and patient-related (subject-related) data.

A subject may refer to a patient receiving healthcare services and, in some implementations, may more generally refer to any individual or entity about which the system obtains data and generates various types of outputs. The subject-related data may include patient-related data (e.g., demographic data, medical history, clinical notes, imaging data, laboratory data, device data, and/or transcripts), and the subject-related information may include any generated or derived outputs based on the subject-related data.

An interaction instance may include a patient visit, appointment, encounter, consultation, check-in process, and/or other interaction between a subject and a service provider, including interactions captured by audio, forms, messaging, and/or device data. The interaction data may include various forms or types of narratives, notes, summaries, transcripts, structured fields, annotations, and/or other generated outputs associated with a given interaction instance between a subject patient and a service provider (e.g., doctor, clinician, nurse). In healthcare implementations, interaction data may include a clinical note (e.g., a SOAP note, history and physical (H&P) note, progress note, discharge summary, or other documentation), and may also include codes, recommendations, and other outputs derived from the interaction instance (e.g., patient visit).

A patient or subject morphology may refer to a physical form or structure of a subject, including anatomy and/or anatomical structures, and in some implementations may include external morphology, internal anatomy, and/or medically relevant structures derived from imaging data. The annotation or exposition may refer to a display element of a graphical user interface (e.g., an overlay of an image annotation) presented in a user interface that contains interaction data and/or other subject-related information; in healthcare implementations, an exposition may include or be implemented as an annotation (e.g., text labels, markers, or other UI elements) mapped to a corresponding portion of the subject's morphology (or anatomy).

Embodiments may perform operations for automatic narrative generation and implement customized patient form integration. A computer may execute the programming for configuring customized subject-intake forms (sometimes referred to as patient-intake forms) or data collection forms. The computer may execute programming a machine-learning model of a machine-learning architecture trained to convert and ingest subject data via the customized subject-intake forms to generate form narratives outputs based on the subject-supplied inputs into the subject-intake forms. In some cases, the computer may ingest or otherwise incorporate the form narratives and other types of subject-related data inputs to generate interaction data (e.g., a clinical note) or other types of AI-generated outputs for a subject or a subject's interaction instance (e.g., patient's visit).

Embodiments may perform operations for detecting discrepancies amongst sources of patient-related data. A computer may execute programming for a machine-learning architecture implementing a machine-learning model for discrepancy detection (“discrepancy detection model”), trained for detecting discrepancies between subject-supplied (sometimes referred to as patient-supplied) data inputs as compared against trusted input sources, such as healthcare provider-supplied inputs (e.g., doctor-supplied data inputs) or stored patient-related data inputs (e.g., EHR data records).

Embodiments may perform operations for generating 3D model visualizations of patient anatomy (i.e., patient morphology) based on patient data inputs, which may be displayed on 3D model user interfaces. A computer may execute programming for a machine-learning architecture implementing a machine-learning model for ingesting patient-related data for a patient from various data sources and generating a 3D model visualization representing the patient anatomy. The computer may further execute programming for generating form narratives or clinical notes (i.e., interaction data) related to portions of the patient's anatomy that the computer may reference and include in the 3D model user interface.

Additionally or alternatively, embodiments may implement 3D model visualization for generating clinical notes. A computer may, for example, generate clinical notes or form narratives using 3D models. The computer generates and references the 3D anatomical models for clinical charting and as an input to the machine-learning models for automatic narrative generation. The 3D model user interface includes and displays an interactive and annotated 3D image, which may be connected to annotation overlays and/or expositions containing text or visual information about the patient anatomy (or subject morphology), such as text of a clinical note (i.e., interaction data) generated by an LLM using patient data captured for a patient visit (i.e., an interaction instance). The 3D models include anatomically correct representations, such as a 3D model of a face with muscular layers and skin. Likewise, the 3D model user interface may accept inputs from a user (e.g., patient, provider) to update portions of the patient data according to the user inputs. The computer may feed the user inputs or computer-generated annotated information charted (e.g., treatment rendered, exams and findings, notes, products used (tracking both cost and inventory), services performed, and/or drawing on the 3D-model to produce bodily location specific complete narratives) on the 3D models into the LLM to generate or update the corresponding portions of clinical notes and/or 3D model visualization.

Embodiments may implement a self-service kiosk device located at a care provider's office. The kiosk includes computing device programmed and configured to function as a self-service terminal and/or a payment terminal for patients during patient visits. The kiosk may be wired or wirelessly networked to other computers of the care provider, which includes administrative software that can control or manage the kiosk. Conventionally, medical offices will offer a self-service terminal to navigate a building or appointments and/or a payment terminal, separate from the self-service terminal. The self-service kiosk described herein combine these functions. Patients can use the self-service kiosk to, for example, sign-in and check-in for an appointment, enter patient-supplied data into patient forms, and make payments, among other features. In some implementations, the self-service kiosk can also seamlessly transition between self-service and staff-controlled administration operating modes. For instance, the provider staff of the medical office can remotely control the self-service kiosk screen to, for example, push or present patient forms at the user interface for the patient to complete or enter payment information for payment requests, among other administrative operations.

### Example System Components

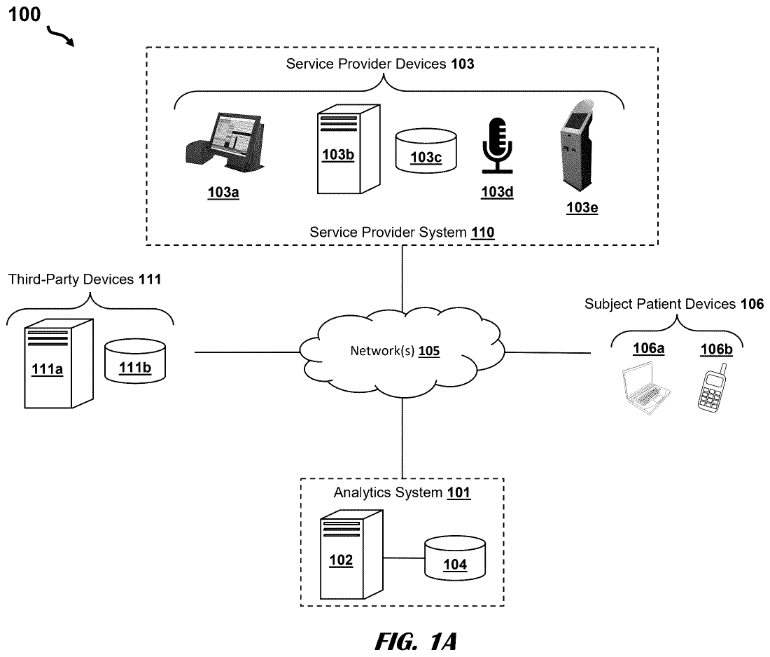

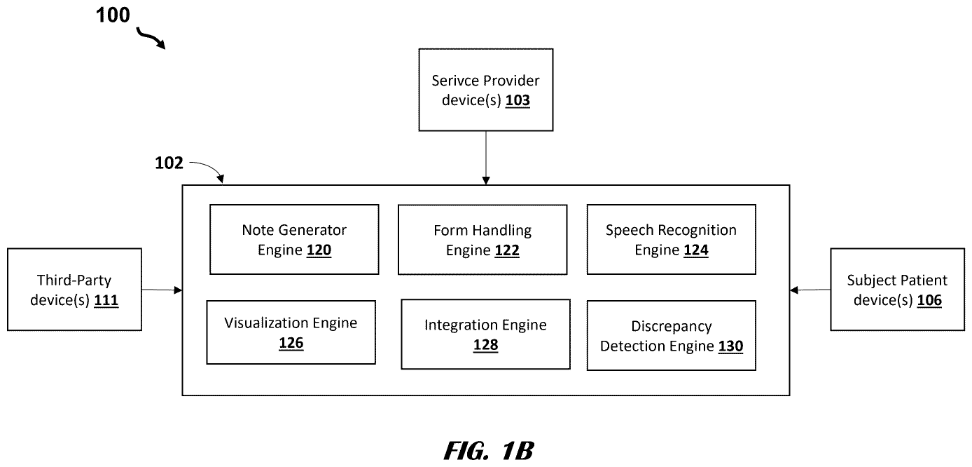

FIGS. 1A-1B show components of a system 100 for capturing various types of data inputs and generating narratives for healthcare notes. FIG. 1A depicts the various device components of the system 100 in a networked environment. FIG. 1B depicts an example collection of executable software engines of analytics servers 102.

Embodiments may comprise additional or alternative components or omit certain components from those of FIGS. 1A-1B, and still fall within the scope of this disclosure. It may be common, for example, to include provider systems and third-party systems with any number of provider devices 103 and third-party devices 111. Embodiments may include or otherwise implement any number of devices capable of performing the various features and tasks described herein. For example, FIG. 1A depicts the analytics server 102 as a distinct computing device from the analytics database 104. In some embodiments, an analytics database 104 may be integrated into the analytics server 102.

Turning to FIG. 1A, the system 100 includes an analytics system 101 for hosting and implementing a clinical documentation system (CDS), a healthcare provider system 110 that includes a network infrastructure of computing hardware and software for providing care services by a care provider (e.g., physician, hospital, laboratory), subject patient devices 106a-106b (generally referred to as subject patient devices 106), and third-party devices 111a-111b (generally referred to as third-party devices 111). The system 100 further includes any number of networks 105 for interconnecting the various components of the system 100. The analytics system 101 includes an analytics server 102 and analytics database 104. The healthcare service provider system 110 includes various healthcare service provider devices 103a-103e (generally referred to as service provider devices 103), including a service provider computing device 103a, service provider server 103b, service provider database 103c, service provider microphone 103d, and self-service kiosk device 103e of the service provider. The subject patient devices 106 include a subject patient computing device 106a and subject patient mobile device 106b. The third-party devices 111 includes a third-party server 111a and third-party database 111b.

### Networks

The networks 105 of the system 100 include hardware and software components for interconnecting the various components of the system 100. The networks 105 may include any number of public networks and/or private networks to interconnect the various components of the system 100. Non-limiting examples of the networks 105 may include Local Area Network (LAN), Wireless Local Area Network (WLAN), Metropolitan Area Network (MAN), Wide Area Network (WAN), and the Internet. The communication over the network may be performed in accordance with various communication protocols, such as Transmission Control Protocol and Internet Protocol (TCP/IP), User Datagram Protocol (UDP), and IEEE communication protocols.

### Patient Devices

A patient device 106 includes a computing device that a patient operates to access and interact with the healthcare-related services of a care provider or analytics system 101 via the networks 105. For instance, the patient devices 106 may launch and execute an application or web browser to access a web-app or webpage hosted at the provider server 103b (or other device of the system 100), where the patient devices 106 transmits patient data as entered by the patient interacting with the web-app or webpage. The patient device 106 may be any computing device comprising hardware components (e.g., processor, non-transitory machine-readable storage media) and software components (e.g., software application(s), web browser), enabling the patient device 106 to perform the various operations and features described herein. Non-limiting examples of patient device 106 may include a patient computing device 106a and patient mobile device 106b, among others.

### Healthcare Provider System and Provider Devices

The provider system 110 represents a network infrastructure comprising physically and logically related software and electronic devices managed or operated for the healthcare provider. The provider system 110 comprises various hardware and software components that generates or otherwise obtains various types of patient-related data or metadata related to the patients, patient devices 106, or contact events for interactions between a patient and the care provider, including patient demographic data, patient-care records or history, and patient-care notes, among other potential types of data.

### Provider Computing Device

The provider computing device 103a includes any computing device allowing a healthcare provider user (e.g., physician, nurse, receptionist, researcher) to access, update, or otherwise interact with patient-related data. For instance, the provider computing device 103a includes software programming for a graphical user interface allowing the provider user to enter patient-related data and display various types of outputs from the analytics system 101, among other operations. The provider computing device 103a may be any computing device comprising hardware components (e.g., processor, non-transitory machine-readable storage media) and software components (e.g., software application(s), web browser), enabling the provider computing device 103a to perform the various operations and features described herein. Non-limiting examples of the provider computing device 103a may include a personal computer (e.g., workstation, laptop), mobile device, or tablet device, among other types of computing devices.

As an example, during a patient visit, a doctor operates the provider computing device 103a to retrieve and review historic patient data from a provider database 103c to prepare for the patient visit and enter new provider-user inputs containing new patient data associated with the patient visit via a user interface at the provider computing device 103a. The doctor may operate a care-related software application executed by the provider computing device 103a, which instructs the provider computing device 103a to access historic patient data in patient data records stored in the provider database 103c or third-party database 111b, such as Electronic Health Record (EHR) data records. For instance, as the patient describes physical state or symptoms and medical history, the doctor may enter provider-user inputs containing this information into the user interface of the provider computing device 103a as provider inputs, which the provider computing device 103a stores into the provider database 103c or other storage device of the system 100 as patient data for the particular patient visit. In some implementations, the computing device 103a allows the doctor to enter or select patient data from predefined fields as provider-inputs of the patient data, such as common conditions, medications, and procedures.

### Provider Server

The provider server 103b executes software programming for receiving patient-related from the provider devices 103 or other components of the system 100 and interacting with the patient devices 106 and analytics server 102 of the analytics system 101. The provider server 103b may be any computing device comprising hardware components (e.g., processor, non-transitory machine-readable storage media) and software components (e.g., software application(s), web browser), enabling the provider server 103b to perform the various operations and features described herein. Non-limiting examples of the provider server 103b may include a personal computer (e.g., workstation, laptop), mobile device, or tablet device, among other types of computing devices. For ease of description and understanding, the analytics server 102 performs the various operations of the machine-learning architectures, but embodiments are not so limited. In some implementations, the provider server 103b may perform certain operations of the machine-learning architectures of the analytics server 102 described herein.

In some implementations, the provider server 103b hosts a web portal or cloud-software programming that receives, stores, and updates patient-related data for the various devices of the system 100. For instance, the provider server 103b receives user inputs from patients or providers, as entered at a graphical user interface of a provider devices 103 or analytics server 102. The provider server 103b may update the patient data in the provider database 103c or send the patient data to a graphical user interface of a provider computing device 103a.

### Provider Database

The provider database 103c may be hosted on a computing device (e.g., server, desktop computer) of the healthcare provider system 110 comprising hardware and software components capable of performing the various processes and tasks described herein, such as non-transitory machine-readable storage media and database management software (DBMS). For instance, the provider database 103c may be hosted and executed by the provider server 103b or other provider device 103 of the healthcare provider system 110. The provider database 103c stores and maintains a comprehensive array of information for healthcare services. This includes patient demographic data, which encapsulates personal details such as name, age, gender, and contact information, among other types of patient data. Additionally, the provider database 103c holds patient-care records that include medical histories, information for past visits, diagnoses, treatments, procedures, and outcomes, among other types of patient data. The patient data of the provider database 103c may include patient-care notes, often written or recorded by physicians or other healthcare professionals, containing the various types of patient data and other types of information, such as observations, recommendations, and follow-up actions for each patient visit. Furthermore, the provider database 103c may contain metadata related to patient devices 106, capturing interactions and contact events between patients and healthcare providers.

In some embodiments, the provider computing device 103a or the provider server 103b executes software programming for training or executing one or more machine-learning architectures. In such embodiments, the provider computing device 103a or the provider server 103b may reference patient data records of the provider database 103c as training data to train a machine-learning architecture. In some cases, the provider database 103c or other database (e.g., analytics database 104) stores training audio signals and associated labels, used by the analytics server 102 or provider server 103b for supervised training of neural networks for speech recognition. The provider database 103c or other database of the system 100 (e.g., analytics database 104, third-party database 111b) contains any number of corpora of training data that are accessible to the analytics server 102 via one or more networks 105. In some embodiments, the analytics server 102 employs supervised training to train the neural network, where the analytics database 104 and/or the provider database 103c contains labels associated with the training audio signals or enrollment audio signals. The labels indicate, for example, the expected data for the training signals or enrollment audio signals. The analytics server 102 may also query an external database (not shown) to access a third-party corpus of training audio signals. An administrator may configure the analytics server 102 to select the training audio signals having varied types of speaker-independent characteristics.

### Provider Microphone

A provider microphone 103d includes any electronic device that captures and converts analog audio waveforms to electrical audio signals. Non-limiting examples of the provider microphone 103d includes a dynamic microphone, condenser microphone, piezoelectric microphone, MicroElectrical-Mechanical System (MEMS) microphone, ribbon microphone, and carbon microphone, among others. The provider microphone 103d includes a transducer that converts the analog audio waveforms into electrical audio signals. A software component of the provider microphone 103d or other device of the system 100 converts the electrical audio signal into a digital audio signal in a data format compatible with the devices of the system 100. For ease of description, the provider microphone 103d is described as generating the digital audio signal containing audio data, though other devices may generate the digital audio signal using the electrical audio signal generated by the provider microphone 103d.

One or more devices of the system 100 (e.g., analytics server 102, provider computing device 103a, provider server 103b) execute software programming of speech recognition engines for analyzing the audio data of the digital audio signal from the provider microphone 103d and identifying content of the audio data, such as the content of a discussion between a care provider and a patient during a patient visit. The speech recognition engine includes software routines of the one or more machine-learning architectures described herein for recognizing the content of the speech within the audio data and generating one or more outputs according to the recognized speech content. Non-limiting examples of the speech recognition engine includes a Text-to-Speech (TTS) engine, an Automated Speech Recognition (ASR) engine, and speaker diarization engine, among others.

The provider microphone 103d may be embedded as a component of another provider device 103. As an example, in some cases, the provider microphone 103d is embedded as a component of the provider computing device 103a. In such cases, the provider computing device 103a can record a provider's voice notes or inputs and execute software of TTS engine to convert the spoken voice notes into text data as new patient-related data, dictated by the provider via the provider microphone 103d.

The provider microphone 103d may be a standalone device that captures and sends audio data to another provider device 103 of the healthcare provider system 110. As example, in some cases, the provider microphone 103d may be situated in a consultation room of a doctor's office and captures ambient speech during a discussion between a patient and doctor. The provider microphone 103d converts the analog audio waveforms of the conversation into electrical audio signals. Software programming associated with the provider microphone 103d digitizes the electrical audio signal into the audio data of the digital audio signal and transmits the audio data to a provider device 103 (e.g., provider computing device 103a, provider server 103b, provider database 103c). As described herein, this digitized audio signal may be analyzed by one or more AI/ML engines to convert the conversation to clinical narratives by mapping the data points within the conversation to one or more clinical narrative sections, such as “SOAP” sections (e.g., (1) Subjective: patient responses, history of present illness (HPI), review of system (ROS); (2) Objective: exams, tests; (3) Assessments: Diagnosis and doctors findings; and (4) Plan: how to remedy and next steps).

### Self-Service Kiosk

A patient self-service kiosk device 103e can be used in a healthcare setting to streamline various administrative tasks and improve patient experience. The kiosk device 103e can be located at the care provider's office and configured to function as both a self-service terminal and a payment terminal. A patient can use the kiosk device 103e to sign in or check in for an appointment, fill out necessary forms, and/or make payments. The kiosk device 103e can be wirelessly connected to other provider devices 103 (e.g., provider computing device 103a, provider server 103b), which includes administrative software that can control or manage the kiosk device 103e. Beneficially, the integration of the patient management operations and payment operations into a single, patient-facing device can reduce the need for multiple separate terminals, thereby optimizing space and resources in the medical office. The use of the kiosk device 103e may also be used to remotely track where the patient is in the practice, how long they have been in the waiting room, track which patients are late (and by how much), track which room patients are in use (e.g., using a combination of a tracking board and a 3D tracking board), and/or track where patients are (including not yet arrived).

A patient can input or output various types of data information through various I/O components of the kiosk device 103e, such as touchscreens, card readers, or barcode scanners, among others. Non-limiting examples of patient inputs to the kiosk device 103e can include patient identification information, appointment details, insurance information, and payment details, among other types of patient related data. Non-limiting examples of potential outputs from the kiosk device 103e can include printed receipts, appointment confirmations, and updated patient records. In at least some examples, the kiosk device 103e can also provide real-time feedback to patients, such as notifying the patients of any missing information or confirming the successful completion of their check-in process. In this example, the kiosk device 103e can enhance the efficiency of administrative tasks and improve the overall patient experience by providing a convenient and user-friendly interface for managing appointments and payments. In yet another example, the kiosk device 103e may be used to scan an insurance card, perform an optical character recognition (OCR) of the insurance card, extract relevant data (e.g., names, date, account identifiers, etc.) from the insurance card, create an insurance profile, run an eligibility check of the insured based on a recommended treatment, and estimate/provide an explanation of payment prior to receiving treatment.

The kiosk device 103e includes computing device programmed and configured to function as a self-service management terminal for patients to manage appointments, which may include operations for checking-in to an appointment, managing appointments, and displaying patient-intake forms at a user interface of the kiosk device 103e for patients to enter patient-supplied input data into the data fields of the patient-intake forms.

A patient can use the self-service kiosk device 103e or other user device (e.g., patient device 106) to manage appointments. This includes requesting an appointment in real-time (e.g., for urgent care or walk-in patients), checking in for appointments, rescheduling or canceling appointments, and receiving appointment reminders and confirmations. The kiosk device 103e can display patient-intake forms on the user interface, allowing the patient to review and update patient data, such as medical history and insurance details before consultation with the healthcare provider. This pre-appointment data entry speeds up the check-in process and provides a data capture channel providing up-to-date patient information for the healthcare provider system 110 and analytics system 101

The kiosk device 103e can support, present, and otherwise display patient-intake forms to capture patient-supplied input data. A patients can interact with the user interface to enter patient inputs to complete patient-intake forms, such as medical history forms, consent forms, medical questionnaires, custom practice-designed-clinical forms, patient forms, and insurance forms, among others, all or some of which may be unique. Additionally, the kiosk device 103e can provide real-time feedback, notifying patients and/or a provider computing device 103a of any missing or incorrect information, when the kiosk device 103e or other device of the system 100 (e.g., provider computing device 103a, provider server 103b, analytics server 102) detects missing or inaccurate information as expected at the patient-intake form.

In addition to appointment management, the self-service kiosk device 103e can function as a payment terminal. A patient can use the kiosk device 103e to provide payment or insurance information at payment user interfaces to, for example, settle co-pays, outstanding balances, and other payments related to healthcare services. In some embodiments, the kiosk device 103e is equipped with various payment hardware and software components, including credit card readers, contactless payment systems, and barcode scanners for insurance or payment cards. This integration of payment operations into the kiosk device 103e reduces the need for separate payment terminals, optimizing space and resources within the healthcare provider system 110 or healthcare provider office.

In some implementations, the kiosk device 103e generates or otherwise displays user interfaces containing real-time notifications or feedback to a patient interacting with a patient intake form, payment user interface, or other types of user interfaces. For instance, a device of the system 100 (e.g., kiosk device 103e, provider server 103b, analytics server 102) may detect missing fields or documentation files for a given intake form and, in response, instruct the kiosk device 103e to present a user interface containing an alert for a patient if any required fields in the form are incomplete or if there is a need for additional documents. In some cases, after the patient completes a check-in process using the kiosk device 103e, the kiosk device 103e can print receipts, appointment confirmations, or other relevant documents.

The self-service kiosk device 103e can transition between self-service operations and staff-controlled administration. This flexibility allows healthcare provider staff to remotely control the kiosk device 103e interface when necessary. For example, provider staff can operate a provider computing device 103a to connect to the kiosk device 103e, where the provider computing device 103a pushes patient forms to the kiosk device 103e for display at the user interface or otherwise instructs the kiosk device 103e to perform certain patient-facing operations, such as a check-in process for the patient to complete the patient forms, a payment process for entering payment information, or provide additional guidance during the check-in process.

The self-service kiosk device 103e is wirelessly connected to other provider devices 103, such as provider computing devices 103a and provider servers 103b. This connectivity allows the patient-supplied input data entered by patients at the kiosk device 103e is immediately transmitted to one or more provider devices 103 or devices of the analytics system 101. By automatically categorizing, mapping, and storing patient data in real-time, the kiosk device 103e facilitates accurate and efficient data management. This integration also allows for the seamless updating of patient records and improves the accuracy of patient information across the healthcare provider system 110. As described below, the analytics system 101 be used to convert these kiosk interactions, conversations, notes, etc. into actual clinical encounter notes.

### Analytics System

The analytics system 101 hosts the CDS for capturing and analyzing patient-related data and generating various types of outputs related to patient care, such as AI-generated patient-care notes and visualizations of the patient, among other types of outputs. The analytics system 101 represents a network infrastructure comprising physically and logically related software and electronic devices managed or operated for a data analytics service provider (also referred to herein as a service provider). The analytics system 101 includes hardware and software components for obtaining the various types of data or information related to a patient (or other subject), including patient demographic data, patient-care records or history, and patient-care notes, among other potential types of data. In some implementations, the CDS (and/or analytics system 101) may be referred to as a data system, and a provider computing device 103a (or other provider device 103) may be referred to as a service computing device used by the service provider to access the services described herein. The devices of the network system infrastructure of the analytics system 101 are configured to provide the intended services for hosting the CDS. The analytics system 101 hosts the CDS as a remote or cloud-based service that performs the various functions, operations, and benefits on behalf of any number of healthcare provider systems 110 or similar healthcare related entities. For instance, physician offices, hospitals, medical researchers, or insurance providers may transmit or upload patient-related data or instructional commands (sometimes referred to as requests) to the CDS of the analytics system 101 via the one or more networks 105, and the CDS of the analytics system 101 may generate and return the outputs or instructional commands (sometimes referred to as responses) to the provider devices 103 via the one or more networks 105.

### Analytics Server

The analytics server 102 includes hardware components (e.g., processors, non-transitory machine-readable memory) and software components (e.g., software code of machine-learning architectures) for implementing the various processes described herein. The analytics server 102 may obtain (e.g., receive, retrieve) and processes patient-related data received from the various types of data sources, such as the analytics database 104, provider devices 103, patient devices 106, and third-party devices 111, among others. Although FIGS. 1A-1B show only a single analytics server 102, the analytics server 102 may include any number of computing devices. For instance, the analytics server 102 may host and include the analytics database 104 or may be in networked communication with another device of the analytics system 101 hosting the analytics database 104. In some cases, the computing devices of the analytics server 102 may provide all or portions of the processes and benefits of the analytics server 102. The analytics server 102 may comprise any number of computing devices operating in a distributed or cloud computing configuration and/or in a virtual machine configuration. Moreover, in some embodiments, functions of the analytics server 102 may be partly or entirely performed by other devices of the system 100, such as the provider devices 103 and/or the patient devices 106.

The analytics server 102 receives, retrieves, captures, or otherwise obtains various types of data related to a patient, where the patient-related data may be obtained as various types of data formats or modalities, such as text data or audio data, among others. The analytics server 102 executes software for ingesting and processing the patient data, including one or more machine-learning architectures, such as an LLM or other neural network architecture, for ingesting and analyzing the patient data and producing various types of AI-generated patient data outputs. Non-limiting examples of the patient data sources and patient data include patient intake forms, Electronic Health Records (EHRs), and voice recordings of patient-doctor interactions, among other relevant types of data and data sources, some or all of which may be converted by the analytics system 101 into clinical narratives, such as for the subjective section within SOAP narratives, History and Physical (H&P) narratives, and other major/formal clinical notes types.

The analytics server 102 includes software programming to execute, invoke, or otherwise perform various operations of one or more machine-learning architectures. These operations may include generating, training, or tuning layers of the machine-learning architectures. The analytics server 102 may train or tune executable layers of a machine-learning architecture into the analytics database 104. The analytics server 102 may further obtain and store, into the analytics database 104, a comprehensive dataset of patient data, which the analytics server 102 may reference in various operations, such as training data for training the machine-learning architectures of the analytics system 101.

The analytics server 102 executes the operations of a machine-learning architecture in several operational phases, including training operations for a training phase and deployment operations at a deployment phase (sometimes referred to as inference, test phase, or testing). The machine-learning architectures of the analytics server 102 ingest and process the various types of input data according to the operational phase. For example, the input data includes training data during a training phase for training parameters of the machine-learning architecture layers or includes input patient data during a deployment phase that the trained machine-learning architecture ingest and process to generate the intended outputs or results.

The analytics server 102 includes software programming for performing operations for analyzing patent data and producing certain types of outputted information or graphical user interfaces for display at provider devices 103 or patient devices 106. For ease of description and understanding, the software operations are described and represented as software engines of the analytics server 102, though embodiments are not so limited. The software programming and functions may have any arrangement and need not be arranged as software engines described herein. Moreover, in some embodiments, the functions of certain software engines described herein may be performed by any number of additional or alternative computing devices of the system 100.

The analytics server 102 may include software programming for providing, on the provider devices 103, clinical recommendations. For example, the analytics server 102 may cross-reference problem lists and family history to provide AI-driven recommendations such as tests to consider, potential future risks (e.g., cancer, diabetes, etc.), and/or allergy risks and preventative recommendations. These recommendations may be displayed, for example, on the patient chart.

Additionally or alternatively, the analytics server 102 may be configured to provide for display on the provider devices 103 a general chatbot to receive questions and instructions to adjust a role-specific context to be used by the analytics server 102. For example, the general chatbot may be integrated with the note generator engine 120 to present a history of questions and answers, edit responses and push them into specific sections of the clinical note, or change a context for the analytics server 102 to use when executing the process and methods described herein. By way of example, the analytics server 102 may receive an indication from a user through the provider devices 103 to change the context between a coder, provider, manager, physical therapist, dietician, medicine manager, surgeon, mental health provider, psychiatrist, esthetician, marketer, accountant, compliance, explainer of benefits, laboratory technician, and/or hormone replacement expert. By changing the context for the analytics server 102, the analytics server 102 may adjust the information provided to the user, such as in graphs, report, illustrations, text, etc.

With reference to FIG. 1B, the analytics server 102 may include and execute a note generator engine 120, form handling engine 122, speech recognition engine 124, visualization engine 126, integration engine 128, and a discrepancy detection engine 130, among others. In some configurations, the respective software engines may execute or invoke software routines of the various machine-learning architectures described herein.

### Note Generator Engine

The note generator engine 120 executes software routines for generating care service-provider notes for patients, such as patient visit notes containing a provider's notes for a given patient visit (also referred to herein as an interaction instance). The note generator engine 120 may generate interaction data for an interaction instance, such as a clinical note, a clinical narrative, and/or other generated text outputs corresponding to the patient visit. The note generator engine 120 includes or invokes a generative machine-learning model (e.g., an LLM) for producing the AI-generated notes. In such implementations, the generative machine-learning model generates output text (e.g., sequences of tokens/strings) conditioned on input data including prompts and patient-related (subject-related) data associated with the interaction instance.

In a training phase, the training process for the generative machine-learning model (e.g., LLM) of the note generator engine 120 involves providing a large and diverse dataset of patient-related training data to the analytics server 102 as the training corpora, which includes various forms of training clinical data. The training data inputs to the generative machine-learning model can include structured and unstructured clinical data, such as patient intake forms, EHRs, and transcripts or audio data for voice recordings of patient-doctor interactions. In some examples, the weights of the generative machine-learning model are updated during backpropagation by comparing the model's predicted outputs against expected data (e.g., actual data or training labels), determining a level of error or loss, and adjusting the parameters to minimize the error or loss. In some cases, a loss layer or loss function calculates a gradient of the loss function with respect to the model parameters and, using optimization algorithms such as stochastic gradient descent, updates the weights or parameters. The output of the training process of the analytics server 102 is a trained generative machine-learning model that can generate accurate and contextually appropriate clinical narratives. The analytics server 102 may store the trained model into the provider database 103c or analytics database 104 and execute the trained model on the analytics server 102 or provider server 103b to generate clinical visit notes based on new clinical health data inputs.

At deployment, the generative machine-learning model (e.g., LLM) of the note generator engine 120 generates outputs representing strings of text (e.g., sequences of tokens) that correspond to clinical visit notes based on various inputs, such as the doctor's inputs to the provider computing device 103a for the visit and clinical health data from the various data sources. The inputs to the generative machine-learning model can include structured and unstructured clinical data, such as patient intake forms, EHRs, and transcripts or audio data for voice recordings of patient-doctor interactions. The outputs from the generative machine-learning model of the note generator engine 120 can include strings of text that represent detailed and accurate clinical visit notes, which can be stored into the patient's medical records of the provider database 103c or other database of the system 100.

The note generator engine 120 may include software routines for various prompt engineering operations to retrieve or generate LLM prompts instructing the LLM for generating patient clinical notes. The LLM is guided by custom prompts that are tailored to the specific specialty and type of practice, including with specialty formats for one or more specialties as well as accounting for common note structures and compliance standards. For example, a provider may operate the provider computing device 103a to access a configuration user interface hosted at the provider server 103b or analytics server 102. The LLM prompts may be stored in a provider database 103c or analytics database 104. The provider may access patient data and enter a provider-generated clinical note, then select an interactive user interface element (e.g., button) to instruct the note generator engine 120 to feed the text of the provider-generated note to the LLM. In some cases, the provider may further configure or select stored prompts or categories of prompts related to the provider's specialty, which are specific to the provider's specialty and the type of clinical note being generated. In such cases, the note generator engine 120 feeds the provider-entered clinical note and the selected prompts to the LLM.

The note generator engine 120 may feed various types of patient-related data to the LLM from multiple sources, such as patient forms, clinical notes, and ambient listening transcriptions. The LLM prompts may instruct the LLM to pull relevant patient-related information from the various sources and generate a comprehensive narrative. For example, during the check-in process, patient responses are captured and transferred to the clinical note, which is then enhanced by the LLM using specific prompt. In some cases, the note generator engine 120 uses the LLM prompts to prioritize the patient-related data based on the data source. For instance, patient-supplied data coming from patient forms may be treated differently than trusted data inputs extracted from the patient data stored in one or more databases (e.g., text of provider clinical notes). The LLM prompt may assign relatively higher priority to, for example, provider-supplied data, such that the LLM of the note generator engine 120 generates a clinical note having relatively less qualifying language. Likewise, the LLM prompt may assign relatively lower priority to, for example, patient-supplied data, such that the LLM of the note generator engine 120 generates a clinical note having relatively more qualifying language.

In some cases, the LLM is instructed through the LLM prompts to avoid generating information that is not present in the patient-related data, thereby avoiding hallucinations. This is crucial in the medical field, as accuracy and reliability of the generated text are critical. In such cases, the LLM prompts explicitly instruct the LLM to use only the provided patient data and avoid assumptions or adding extraneous information. In some cases, the LLM prompts enable or tune the LLM to incorporate medical terminology and context. For example, the LLM uses the LLM prompts to understand the context of the patient's medical history, current symptoms, and treatment plans, among other types of data inputs. In some cases, the LLM processes the input data and generates a clinical note, that includes a suggested CPT code based on the recognized medical procedures and diagnoses (e.g., ICD codes, such as ICD-10 codes) in the patient data inputs. For instance, the LLM may be trained to recognize patterns in the training patient clinical data to suggest appropriate CPT codes based on the content of the clinical notes during the deployment phase. In some embodiments, the LLM is configured to return an error and/or note in the event that one or more of the previous outputs of the LLM does not meet standards for a billing level and then provides a suggestion on whether to adjust the error-identified format/information (e.g., upping a code of the note and/or adding additional information) such that the output conforms to the various compliance requirements. This allows for an efficient flow of information between clinical AI models and RCM systems within a healthcare system to expedite claim creation/payment with reduced claim rejections.

The note generator engine 120 may execute optical character recognition (OCR) to recognize text from a provider's handwritten notes (e.g., on a diagnostic scan, a note, etc.) and extract relevant data from the handwritten information to be used by the LLM to generate the AI-generated note. The recognized data may be mapped to known/desired/compatible formats to be used in the revenue cycle management system. In some embodiments, the analytics system 101 may generate follow-up prompts to display on the provider devices 103 for the user to respond to. In some instances, the prompt may request additional information related to the handwritten note or to verify the OCR scan. In some embodiments, the note generator engine 120 recommends or finalizes codes based on the OCR scan of handwritten notes.

The analytics system 101 may use custom mapping of unstructured data based on keywords to covert the unstructured data to structured data. For example, a suggested CPT code is mapped to a coding template to transfer automatically to the claim. In clinical settings, keywords such as “subjective,” “HPI,” “ROS,” whether spelled out or abbreviated, automatically map LLM outputs to the Subjective clinical section, subject to the provider's approval and sign-off.

The analytics system 101 may also convert detailed narratives into “clinical summaries” (e.g., short form notes with codes). This may be useful in clinical settings in which clinical summaries are more common, such as worker's compensation or other legal situations, as well as physical medicine. The analytics system 101 formats the data into simple narratives, such as in bulleted format with preselected or AI-suggested codes. In some embodiments, a discharge summary and patient education materials are automatically generated using the data from the note, intake, patient chart or other encounter.

In some embodiments, the note generator engine 120 may generate an LLM prompt that instructs the LLM to generate a binary response based upon whether a user (e.g., doctor, patient) is requesting a full clinical note, asking a general question, or for an edit to an existing note. A full clinical note is mapped within a desired format (based on selection, e.g., SOAP), and returns various types of data for a clinical note to be generated or reviewed. A question may be rendered in a chat-style, free text, wherein the user can select at least a portion of that response and instruct the user interface to add the outputted response to a clinical narrative template as desired.

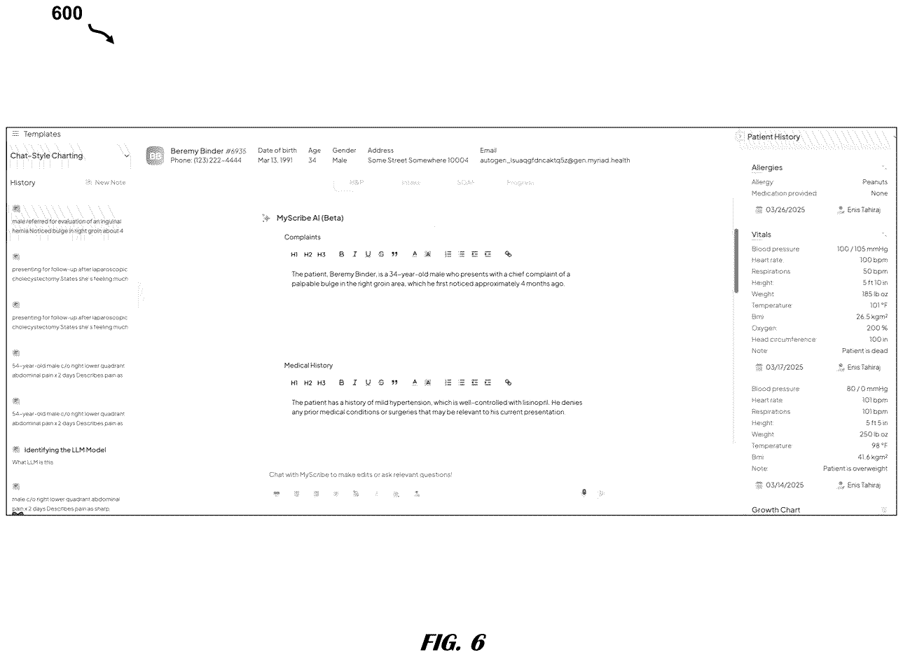

As shown in FIG. 6, a graphical user interface 600 displays an example of “mapped” data, using keywords and the LLM or other machine-learning models to dissect the content and post to a subsection of the clinical note.

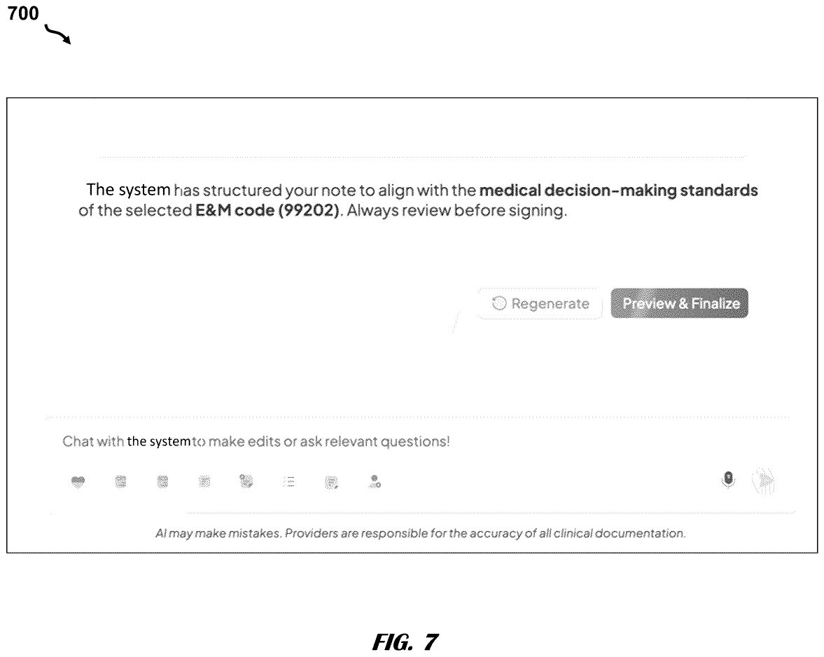

As shown in FIG. 7, a graphical user interface 700 displays a “chat style” response that is not automatically included in a note, but includes graphical user interface elements that display LLM-generated responses to questions, compliance, and coding tips and more.

### Form Handling Engine for Custom Forms and Templates

The form handling engine 122 provides adaptable templates that can capture nuances of individual patient encounters. The flexibility of the form handling engine 122 provides flexible data gather and comprehensive clinical documentation. The same or different AI/ML model trained for automatic narrative generation (e.g., the LLM of the note generator engine 120) can be trained to convert and ingest customized patient intake forms to generate simple narratives for patient intakes notes. This may include, by way of example, converting checkboxes, multi-select fields, and/or open text fields into structured sentences by reversing the question to present the answer as a simplified narrative statement. The form handling engine 122 may ingest and incorporate these narratives of the intake notes, along with other types of data inputs, into a clinical note or other AI-generated outputs for the patient or a patient's visit. This may include, for example, producing more complex narratives than the earlier generated simple narratives and drawing connections between other disparate data.

In some implementations, the form handling engine 122 includes software routines allowing a healthcare provider to generate and map customized intake-form templates via a graphical user interface to certain aspects of healthcare diagnosis or treatments, thereby configuring the machine-learning architecture of the form handling engine 122 to generate narratives that are tailored to the specific needs and preferences of the providers.

In some embodiments, the form handling engine 122 includes a form configuration portal hosted on the analytics server 102. The form configuration portal includes a graphical user interface and software programming allowing healthcare providers to create, customize, and manage customized patient intake forms, such as a patient vaccine forms. The portal provides a graphical user interface that allows the provider to design and personalize intake form templates. Utilizing drag-and-drop functionalities, providers can add, remove, and rearrange form fields to suit specific requirements or provider specialties. The provider may choose from a library of pre-existing templates or create new forms. A form template can be customized to include specific data fields relevant to the patient's visit, such as demographic information, medical history, and vaccine details, or the provider's specialty. In this way, the portal of the form handling engine 122 allows the provider to tailor the templates to the needs of various clinical scenarios.

In some cases, the form handling engine 122 integrates real-time data from EHRs and other clinical data sources into a patient intake form. As an example, as the patient enters a patient intake form, the form handling engine 122 may automatically pull relevant information from the various data sources to complete the data fields of the patient intake fields, minimizing manual data entry and reducing the risk of errors.

As with the note generator engine 120, the machine-learning model of the form handling engine 122 may include a transformer-based model (e.g., LLM, BERT, GPT), for handling complex language tasks and generating coherent text. In an example, the machine-learning model can be implemented by analytics server 102 to handle customized forms by analyzing the input data and generating appropriate outputs. The inputs to the machine-learning model of the form handling engine 122 can include various types of structured and unstructured data, such as data fields of patient intake forms, EHRs, and other relevant clinical data. In some examples, the outputs from the machine-learning model of the form handling engine 122 can be structured data entries, generated narratives, or other forms of processed information that can be integrated into other data records of the patient or other software components of the CDS engines of the analytics server 102.

The training process for the machine-learning model involves providing a large and diverse dataset as input, which includes various forms of clinical data. In this example, the output of the training process is a model that can accurately process customized forms and generate narratives based upon the patient-supplied data inputs (entered as user interfaces to the patient intake forms) or detect missing or incorrect data inputs to the intake forms. In some examples, the computer executes a loss function that updates the weights of the machine-learning model during backpropagation by comparing the model's predictions to the actual data and adjusting the parameters to minimize the error. In at least some examples, the loss layers of the machine-learning architecture determine a gradient of the loss function with respect to the model's parameters and using optimization algorithms, such as stochastic gradient descent, to update the weights accordingly. The trained machine-learning model can then be deployed on the server 102 to handle customized forms based on new clinical data inputs from provider computing device 103a, kiosk device 103e, or patient devices 106.

At deployment, as patients or healthcare providers enter data into the custom templated forms, the form handling engine 122 can generate narratives in real-time. For instance, a trained LLM may generate text narrative of the competed form based on the provided information. In this example, the form handling engine 122 can be set to enhance these simple narratives using the LLM, automatically transforming the narratives into more detailed and coherent narratives. In some examples, the form handling engine 122 can provide real-time feedback, such as notifying providers of any missing information or confirming the successful completion of the documentation process. This approach improves the overall efficiency and accuracy of clinical documentation by providing a convenient and user-friendly interface for managing customized templates.

For instance, when a patient has completed an intake form, the form handling engine 122 invokes the LLM or the note generator engine 120 to produce detailed narratives based on the provided information in the patient intake form and other data sources. In some case, the narratives generated for the form intake may be generated as structured data for inclusion into a portion of patient clinical notes or ingested by the note generator engine 120 for generating the clinical notes for the patient clinical note for the patient visit. In some cases, the form handling engine 122 generates notification outputs for display at a user interface of the patient devices 106 the provider devices 103, based on data entered into the data fields of the intake form and other patient-related data from the other data sources. For example, the form handling engine 122 can detect missing or incomplete fields of the patient intake form and notify the provider of any missing information or confirm the successful completion of the form.

As an example, the provider may access the portal to configure a vaccine form. To configure a vaccine intake form, the prover operates the provider computing device 103a to access the form configuration portal of the form handling engine 122 on the analytics server 102. The provider selects a pre-existing vaccine template or creates a new template from scratch. The provider may interact with configuration features of the portals, such as a drag-and-drop functionality to add relevant fields, such as patient name, date of birth, vaccine type, dosage, and administration site. The form handling engine 122 instructs the analytics server 102 access various other data sources, such that form handling engine 122 may integrate real-time data from the other data sources, such as EHRs, to auto-populate the types of fields. In some implementations, the form handling engine 122 may incorporate patient-related into the data fields with existing patient information for a particular patient. The analytics server 102 may store the intake template form into the analytics database 104 or provider database 103c for future use or generate the intake form for immediate patient documentation. At the time of patient intake, the patient operates the patient devices 106 or kiosk device 103e to access one or more patient intake forms, which are presented at the user interface of the patient device 106 or kiosk device 103e, allowing the patient to enter information in the data fields via the graphical user interface. Upon completion, the machine-learning model of the form handling engine 122 may generate a detailed narrative of the patient's intake forms using the note generator engine 120.

### Speech Recognition Engine

The speech recognition engine 124 of the analytics server 102 captures audio data from conversations between patients and healthcare providers. The speech recognition engine 124 includes a machine-learning model of the machine-learning architecture, such as ASR or TTS, for processing spoken language, identifying content in the audio data, and converting the content of the audio data into text data or other types of outputs.

When a patient and healthcare provider engage in a discussion, the speech recognition engine 124 actively listens and captures the audio data by obtaining and analyzing audio data from the provider microphone 103d. The speech recognition engine 124 executes the machine-learning model trained specifically for medical terminology and conversational nuances. The speech recognition engine 124 includes an ASR or TTS that, for example, converts the audio data into a transcription of text data. In some embodiments, after the audio data is transcribed, the speech recognition engine 124 may execute natural language processing (NLP) techniques to further refine the text. This processing includes identifying key medical terms, patient symptoms, diagnoses, and treatment plans mentioned during the conversation. The refined text is then structured into coherent and comprehensive patient clinical notes.

A machine-learning model of the speech recognition engine 124 may include an ASR engine for generating accurate text transcriptions of audio data, which may include, for example, a doctor's spoken notes or the voice audio communications between the patient and the doctor during a patient visit. As an example, an ASR engine can be implemented by the speech recognition engine 124 to identify particular content in the audio signal and convert spoken language into text. In this example, the ASR engine generates the text transcription of conversations, capturing the nuances and complexities of medical terminology used during patient visits.

The inputs to the machine-learning models can include audio data of audio recordings containing voice-speech of patient and doctor interactions captured at the provider microphone 103d, which are processed by the ASR engine of the speech recognition engine 124 to generate text transcriptions. The outputs from the ASR engine may include the text transcription representation of the spoken interaction, which the machine-learning architectures of the analytics server 102 may store into the patient data of the provider database 103c or analytics database 104. The various machine-learning architectures of the analytics server 102, such as the note generator engine 120, may ingest the text transcriptions produced by the speech recognition engine 124 as an input for generating the clinical note, among other potential outputs of the machine-learning architectures.

During a training phase for the ASR engine (or other machine-learning architecture of the speech recognition engine 124) involves providing a large and diverse training dataset of medical audio recordings as training input, which includes various accents, speech patterns, and medical terminologies. A loss layer or loss function of the analytics server 102 updates or tunes weights or parameters of the ASR engine during backpropagation by comparing predicted output transcriptions against the expected outputs (e.g., training labels, actual text) and adjusting the parameters to minimize the error or loss. The loss function may, for example, calculate a gradient with respect to the model's parameters and using optimization algorithms, such as stochastic gradient descent, to update the weights according to the level of error or loss. The speech recognition engine 124 may execute and deploy the trained ASR engine generate text transcriptions of voice audio (e.g., doctor spoken notes, patient and doctor interactions). The output of the training phase includes the trained layers of the machine-learning architecture for transcribing medical conversations. The analytics server 102 may store the trained speech recognition machine-learning model of the speech recognition engine 124 (e.g., ASR engine) into the analytics database 104 or the provider database 103c.

In some embodiments, the training phase of the speech recognition model of the speech recognition engine 124 begins with a corpus of training audio data of training audio recordings, which may be stored in or obtained from various databases of the system 100. The training audio recordings include voice speech containing, for example, various accents, speech patterns, and medical terminologies to provide the speech recognition engine 124 with a comprehensive understanding of the language used in clinical settings. The analytics server 102 may perform various preprocessing operations on the audio data of the training dataset to, for example, remove any noise or irrelevant audio data, and segment the audio signal data into smaller units, such as phonemes or words, which analytics server 102 may associated with corresponding text transcriptions or training labels.

The speech recognition model of the speech recognition engine 124 may be initialized with a set of parameters, which may include random values at the beginning of the training phase. The configurations of the speech recognition model, such as a number of layers and size of each layer, may be preconfigured or adjusted at the training phase. In some implementations, the analytics server 102 trains the speech recognition model using supervised learning or semi-supervised learning, where the speech recognition model learns to map the audio inputs to corresponding text outputs. During training, the loss function of the speech recognition model compares the predicted outputs against the expected outputs (e.g., training labels, actual text transcriptions), and computes the level of error or loss. The analytics server 102 updates or tunes the weights of the speech recognition model during backpropagation by calculating the gradient of the loss function with respect to the speech recognition model parameters. In some implementations, the analytics server 102 implements optimization algorithms, such as stochastic gradient descent, to adjust the weights to minimize the error. The analytics server 102 performs this iterative training process until the speech recognition model achieves a satisfactory level of error or loss, according to a training threshold.

During a deployment phase, the trained speech recognition model of the speech recognition engine 124 can then be deployed in the analytics server 102 to transcribe audio data from the provider microphone 103d, such as voice speech for a patient and doctor interaction or doctor's spoken notes. As an example, when a patient visits a doctor's office for a routine check-up, the doctor and the patient engage in a conversation about the patient's symptoms, medical history, and any concerns they might have. A trained ASR engine of the speech recognition engine 124, implemented by a server 102, is activated to capture and transcribe the spoken interactions captured by the provider microphone 103d. The speech recognition engine 124 processes the audio input, which includes the doctor's questions and the patient's responses, and generates a text transcription of the conversation.

### Integration Engine

The integration engine 128 of the CDS hosted in the analytics system 101 can capture and integrate various types of data, including patient demographic data, patient-care records, clinical notes, and metadata related to patient interactions with healthcare providers. The analytics server 102 implements the integration engine 128 for data-integration operations across the various types of data containing patient-related data, which includes integrating or normalizing the various types of data from any number of data sources of the system 100 and may be used by the other components of the analytics server 102.

The integration engine 128 includes application programming interfaces (APIs) to facilitate data retrieval from external systems from the analytics system 101. The integration engine 128 can pull or retrieve patient-related data from various sources, including patient forms, past clinical notes, patient charts, macros, and EHR records from various databases. For instance, the integration engine 128 can pull or retrieve data from various types of data sources, including EHR records in third-party databases 111b or provider databases 103c or other types of data inputs described herein. The operations of the integration engine 128 harmonizes various data sources, such as lab results, imaging, and patient histories. This integration engine 128 reduce the need for redundant data entry and minimizing discrepancies within patient records. The integration engine 128 can also capture data from patient devices, such as wearable health monitors, and third-party devices 111, including lab equipment and imaging systems. In some examples, the integration engine 128 can aggregate data from multiple sources within the provider system 110, to generate a comprehensive view of the patient's health information. In at least some examples, the integration engine can also capture data related to patient appointments, medication lists, and treatment plans, providing a holistic view of the patient's healthcare journey. The integration engine 128 can retrieve data in response to real-time requests received via the provider computing device 103a or patient devices 106, or at different intervals, such as scheduled intervals or periodic intervals.

### Visualization Engine

The visualization engine 126 of the analytics server 102 includes software programming for generating visual representations of a patient anatomy (or, more generally, a subject morphology) and medical condition annotations, according to the various types of data inputs for display in a graphical user interface at the provider computing device 103a or patient device 106. For instance, CDS hosted on the analytics server 102 provides 3D-modeling aspects for creating detailed and accurate representations of the patient anatomy and medical conditions. These 3D models provide visual aids for healthcare providers or patients to better understand and document patient conditions. The 3D models may also be interactive, allowing the provider to view additional information or interactively provide additional information as provider inputs (also referred to herein as service-supplied inputs) for additional patient-related data.

The visualization engine 126 generates an interactive 3D-model user interface using the various types of patient data from the integration engine 128. The integration engine 128 can capture and integrate, for example, patient demographic data, patient-care records, clinical notes, and metadata related to patient interactions with healthcare providers. The integration engine 128 can also capture data from patient devices 106, such as wearable health monitors, and third-party devices 111, including lab equipment and imaging systems. The integration engine 128 can aggregate the patient-related data from the multiple sources within the provider system 110.

The visualization engine 126 can generate the interactive 3D-model user interface for a patient based on the patient data. The visualization engine 126 can use the aggregated patient data from the integration engine 128 to create detailed and accurate 3D models of patient anatomy and medical conditions. The 3D-model user interface can be displayed on a provider computing device 103a, allowing the healthcare provider to interact with the 3D model visualization. The visualization engine 126 can generate or otherwise include annotations based on the clinical notes generated by the note generator engine 120 or other types of patient-related data. These annotations can provide additional context and information, enhancing the utility of the 3D model visualization for clinical documentation and decision-making.

In an example embodiment, the visualization engine 126 can receive inputs from a doctor interacting with the 3D-model user interface at the provider computing device 103a. In this example, the doctor can use the interface to manipulate the 3D model visualization, add or modify the annotations, and/or input additional data. The visualization engine 126 can process these additional inputs and update the 3D model and annotations accordingly. The visualization engine 126 may provide updated information to the integration engine 128 or other component of the analytics server 102, and the updated information can be integrated back into the patient's records in the various databases, such that the clinical documentation and patient-related data remains accurate and updated.

In an example embodiment, the visualization engine 126 can implement various machine-learning models to generate a 3D model interface based on patient-related data. In this example, the machine-learning models can include convolutional neural networks (CNNs) for processing and interpreting medical imaging data, such as MRI or CT scans. In some examples, the visualization engine 126 can use generative adversarial networks (GANs) (an example of a generative machine-learning model) to create realistic 3D models of patient anatomy by learning from a large dataset of medical images. In at least some examples, the visualization engine 126 can implement recurrent neural networks (RNNs) to analyze time-series data from patent data, such as data produced by wearable health monitors, and integrate this information into the 3D model.

In an example embodiment, the visualization engine 126 can also use autoencoders to compress and reconstruct high-dimensional patient data, enabling efficient storage and retrieval of 3D models. In this example, the visualization engine 126 can employ transfer learning techniques to leverage pre-trained models and adapt them to specific medical applications. In some examples, the visualization engine 126 can use reinforcement learning to optimize the generation and manipulation of 3D models based on training labels and feedback from healthcare providers, entered at the provider computing devices 103a.

The visualization engine 126 can integrate these machine-learning models to create the interactive 3D-model user interface that provides a comprehensive and accurate representation of patient anatomy and medical conditions. In this example, the engine can continuously update the 3D model based on new patient data.

In some implementations, the visualization engine 126 can overlay visual annotations on the 3D model user interface based on the text notes generated by the note generator engine 120. In such implementations, the visualization engine 126 can receive clinical notes and/or form narratives generated by the note generator engine 120 and/or the form handling engine 122, which include detailed information about the patient's condition, treatment, and other relevant data. The overlay display elements may include or be implemented as expositions (e.g., overlays) containing interaction data (e.g., clinical notes) associated with corresponding portions of the patient anatomy (or subject morphology). For instance, the visualization engine 126 is trained to parse the clinical notes and identify clinical information and map the clinical medical information to specific anatomical structures or regions within the patient anatomy represented in the 3D model. In some implementations, the visualization engine 126 executes software routines for natural language processing (NLP) techniques that extract the clinical information from the text notes and generate the corresponding annotations.

As an example, the visualization engine 126 can generate an interactive 3D-model user interface for a patient that includes the annotations and/or expositions. In this example, the expositions (e.g., overlay UI elements) can be overlaid on the 3D model, providing visual markers and labels that correspond to the information in the clinical notes (i.e., interaction data). In some examples, the annotations/expositions can include text labels, arrows, and other visual indicators that highlight specific areas of interest within the 3D model. In at least some examples, the annotations/expositions can be interactive, allowing healthcare providers to click on them to view additional details or related information. The visualization engine 126 can display the 3D-model user interface with the annotations on the provider computing device 103a of the doctor, such that the doctor can interact with the 3D model, manipulate the view, and explore the annotated regions to gain a better understanding of the patient's condition. In some examples, the visualization engine 126 can also receive inputs from the doctor, such as adding or modifying annotations, adjusting the view, or inputting additional data.

In some implementations, the analytics server 102 and/or the provider server 103b execute software programming of the visualization engine 126 to generate and present the interactive 3D model user interface to generate clinical notes based on user inputs entered via the 3D model user interface. The note generator engine 120 or form handling engine 122 may, for example, generate clinical notes or input-form narratives using data entered into a 3D model user interface. The visualization engine 126 generates the 3D anatomical models and receives user inputs (e.g., provider-supplied inputs, patient-supplied inputs) containing patient-related data via the 3D model user interface, which the visualization engine 126 stores into a storage location as updated patient-related data. The note generator engine 120 or form handling engine 122 may ingest these user inputs to generate the clinical notes, form inputs, or form narratives, referencing these user inputs as potential inputs to the corresponding machine-learning models of the note generator engine 120 or form handling engine 122. In this way, the 3D model user interface includes and displays the interactive and annotated 3D image, which may be connected to annotations overlays containing text or visual information about the patient anatomy, such as text of a clinical note generated by an LLM using patient data captured for a patient visit, and the 3D model user interface may accept inputs from a user (e.g., patient, provider) to update portions of the patient data according to the user inputs.

### Discrepancy Detection

The discrepancy detection engine 130 of the analytics server 102 includes software programming for a machine-learning model trained to identify discrepancies between patient-supplied inputs and provider-supplied inputs for the patient-related data. The patient-supplied inputs may originate from various devices, such as patient devices 106, kiosk device 103e, or provider microphone 103d during a patient visit. The discrepancy detection engine 130 can receive patient-supplied inputs from various data sources or types of data, such as patient forms, patient devices 106 (e.g., patient computing device 106a, wearable health monitors), and patient-entered data during check-in processes at the kiosk device 103e, among others. The provider-supplied inputs may originate from various devices, such as provider devices 103. The discrepancy detection engine 130 can receive the provider-supplied inputs from various data sources or types of data, such as the clinical notes, diagnostic reports, and treatment plans entered by healthcare providers, among others. The discrepancy detection engine 130 can compare the patient-supplied inputs against the provider-supplied inputs to identify any inconsistencies or discrepancies.

In some embodiments, the discrepancy detection engine 130 includes software routines of a machine-learning model programed and trained to analyze the patient-related data and detect patterns or anomalies that indicate discrepancies. For instance, the discrepancy detection engine 130 can employ NLP techniques to interpret and compare textual data from patient and provider inputs. In some examples, the machine-learning models of the discrepancy detection engine 130 can also use statistical methods to identify deviations between the patient-supplied inputs and the provider-supplied inputs. The discrepancy detection engine 130 may detect and flag or tag any identified discrepancies when the comparison exceeds a threshold level of distance or difference. The discrepancy detection engine 130 may generate discrepancy alerts indicating a discrepancy for the healthcare provider to review and address the inconsistencies and present the discrepancy alert to the provider via a graphical user interface of the provider computing device 103a.

The discrepancy detection engine 130 can implement various machine learning models to identify the discrepancies between patient-supplied inputs and provider-supplied inputs. In some cases, the discrepancy detection engine 130 may, for example, implement NLP models, such as Bidirectional Encoder Representations from Transformers (BERT), to analyze and compare textual data from patient and provider inputs. In some cases, the discrepancy detection engine 130 can employ convolutional neural networks (CNNs) to process and interpret structured data inputs from the patient and provider, such as patient forms and clinical notes. In some cases, the discrepancy detection engine 130 can use recurrent neural networks (RNNs), including Long Short-Term Memory (LSTM) networks, to analyze time-series data from patient devices 106, such as wearable health monitors and other patient devices 106.

In some implementations, the discrepancy detection engine 130 can also use anomaly detection models, such as Isolation Forests or One-Class Support Vector Machines (SVMs), to identify unusual patterns or deviations in the data that may indicate discrepancies. In such implementations, the discrepancy detection engine 130 can employ ensemble learning techniques, such as Random Forests or Gradient Boosting Machines (GBMs), to combine the outputs of multiple models and improve the accuracy of discrepancy detection. In some examples, the discrepancy detection engine 130 can use transfer learning to leverage pre-trained models and adapt them to the specific context of patient and provider inputs.

In some implementations, the discrepancy detection engine 130 can continuously update and refine the machine-learning models based on new patient-related data from the patient or provider. In some cases, the discrepancy detection engine 130 can generate a discrepancy alert when a discrepancy is detected, allowing the provider to review and address the inconsistency.

As an example, the discrepancy detection engine 130 can use a machine-learning model to detect discrepancies between patient-supplied inputs and provider-supplied inputs by calculating the difference or distance between the two sets of inputs. In this example, the engine can receive patient-supplied inputs, such as self-reported symptoms, medical history, and wearable device data, and provider-supplied inputs, such as clinical notes, diagnostic results, and treatment plans. Optionally, the analytics server 102 may preprocess the inputs to standardize and normalize the input patient-related data. The machine-learning model can use techniques to quantify the difference between feature vectors representing the patient-supplied inputs and the provider-supplied inputs, such as cosine similarity, Euclidean distance, or other distance metrics. The discrepancy detection engine 130 can calculate a discrepancy score that represents the degree of difference between the two feature vectors. In some examples, the discrepancy detection engine 130 can compare the discrepancy score against a predefined discrepancy detection threshold. In some cases, the discrepancy detection engine 130 generates a feature vector representing the patient data stored in the one or more databases, such as EHR data stored in or pulled from a provider database 103c or third-party database 111b. The discrepancy detection engine 130 can calculate an additional or alternative discrepancy score that represents the degree of difference between the feature vectors for the patient-supplied inputs and the feature vector representing the stored patient data. If the discrepancy score exceeds the threshold, the discrepancy detection engine 130 detects a discrepancy and flags the patient-supplied input as a potential discrepancy.

### Analytics Database

The analytics database 104 stores various types of data or information used for training, tuning, or deploying the various executable engines or machine-learning architectures of the analytics server 102. The analytics database 104 may be hosted on any computing device (e.g., server, desktop computer) comprising hardware and software components capable of performing the various processes and tasks described herein, such as non-transitory machine-readable storage media and database management software (DBMS).

The analytics database 104 or other databases of the system 100 (e.g., provider database 103c, third-party server 111a) may store one or more corpora of training data (e.g., training audio signals, training patient data, training metadata), accessible to the analytics server 102 via the one or more networks 105. In some embodiments, the analytics server 102 may employ supervised training to train the one or more machine-learning models of the machine-learning architecture(s) discussed herein. In such embodiments, the analytics database 104 and/or other databases contain training labels associated with the training data. For a particular machine-learning model, the labels indicate, for example, the expected data or expected outputs when the machine-learning model is applied to the training data. The analytics server 102 may also query an external database (e.g., provider database 103c, third-party database 111b) to access additional or alternative training data.

### Example Processes

### Generating a Narrative for a Clinical Note

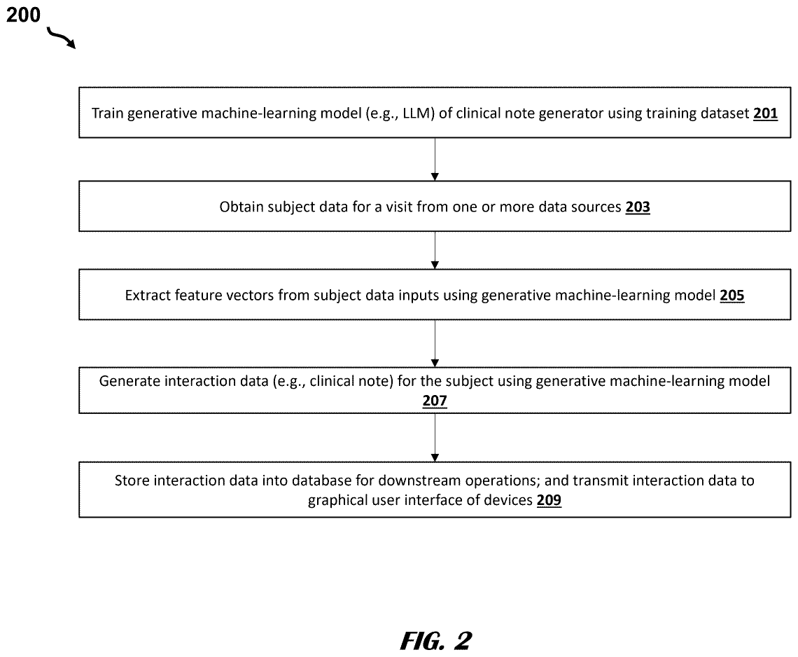

FIG. 2 shows a flowchart of operations of a computer-implemented method 200 for implementing a generative machine-learning model (e.g., an LLM) for generating textual clinical notes for a patient using any number of inputs containing patient-related data. A computing device (e.g., analytics server 102, provider server 103b) executes software programming (e.g., note generator engine 120) for ingesting various types of patient-related data and generating clinical notes via conditional text generation. For ease of description and understanding, the operations of the computer-implemented method 200 are described as being performed by a computer, though embodiments may be implemented using any type of computing device and/or may be implemented using multiple computing devices.

In operation 201, the computer trains a generative machine-learning model (e.g., an LLM) of a clinical note generator (e.g., note generator engine 120) using a training dataset. The computer obtains (e.g., receives, retrieves) training input data from various types of data sources for a plurality of training patients or training data records. The training data can include structured and unstructured clinical data, such as patient intake forms, electronic health records (EHRs), and transcripts or audio data from voice recordings of patient-doctor interactions. Optionally, the computer performs operations for preprocessing the training data to standardize and normalize the various training data records. Non-limiting examples of the preprocessing can include tokenization, removal of irrelevant information, and conversion of audio data to text, among others.

The generative machine-learning model (e.g., the LLM) can be initialized with pre-trained weights or parameters, which can be fine-tuned using the collected training dataset. The computer feeds the training data into the generative machine-learning model and uses backpropagation to update the model's weights. A loss function compares the model's predicted outputs (e.g., next-token predictions in a sequence) against expected data (e.g., actual data or training labels) to determine a level of error or loss. In at least some examples, a loss function calculates the gradient of the loss with respect to the model's parameters, and optimization algorithms, such as stochastic gradient descent, can be used to update the weights to minimize the error or loss.

The computer may train the generative machine-learning model (e.g., LLM) using a training dataset including text of medical terminology, which may be associated with training labels indicating the medical terminology. In some cases, the training labels indicate particular medical specialties associated with the particular medical terminology in the training dataset. The generative machine-learning model tokenizes the text, which includes parsing the text into smaller units or tokens, such as words or subwords, and encodes the tokens into numerical representations, features, or feature vectors that the model can process. During training, the model is trained to generate output text conditioned on clinical context in the input data (e.g., by learning to predict the next token in a sequence based on preceding tokens). The loss function adjusts the parameters or weights of the model to minimize a difference between predicted outputs and expected outputs (e.g., training labels, actual text in training dataset).

In some embodiments, after initial training operations, the computer may fine-tune the LLM using a more specialized dataset that focuses on specific medical terminology or specialties. The fine-tuning adjusts weights or parameters of the LLM to generate more accurate and contextually relevant clinical notes for particular medical fields.

The computer may execute the training operations iteratively, with multiple epochs of training to until the LLM converges to an optimal set of parameters. The computer uses the loss function to evaluate the LLM periodically during training operations to assess the LLM performance and tunes or adjusts the parameters or weights of the LLM to improve the level of error or loss. In some implementations, the computer performs the evaluation using a validation dataset to measure the LLM's accuracy and generalization capabilities. The computer executes the training operations until the LLM achieves a satisfactory level of performance, as determined based upon the level of error or loss satisfying a training threshold. The computer may store the trained LLM into a database (e.g., provider server 103b, analytics database 104).

The computer includes software routines for various prompt engineering operations to retrieve or generate LLM prompts instructing the LLM for generating patient clinical notes. The LLM is guided by custom prompts that are tailored to the specific specialty and type of practice. For example, a provider may operate the computer (e.g., provider computing device 103a) to access a configuration user interface hosted at the computer or other computing device (e.g., provider server 103b, analytics server 102). The LLM prompts may be stored in and retrieved from a database (e.g., provider database 103c, analytics database 104). For instance, the provider may access patient data and enter a provider-generated clinical note, then select an interactive user interface element (e.g., button) to instruct the computer to feed the text of the provider-generated note to the LLM. In some cases, the provider may further configure or select stored prompts or categories of prompts related to the provider's specialty, which are specific to the provider's specialty and the type of clinical note being generated. In such cases, the computer feeds the provider-entered clinical note and the selected LLM prompts to the LLM.

As an example, when a patient visits a provider for an appointment, the patient may operate a self-service kiosk or computing device to perform a check-in process. During the check-in process, the patient fills out a form on the kiosk, providing patient-supplied information about current symptoms, medical history, and any recent changes in condition. This patient input data is transmitted to and received by the computer and stored in the one or more databases. The computer programming (e.g., note generator engine 120) identifies a type of clinical note that needs to be generated, such as a follow-up visit clinical note. The computer then selects an LLM prompt that is specifically designed for follow-up visits in the provider specialty. The LLM prompt includes instructions for the LLM to focus on the patient's current symptoms, any changes since the last visit, and relevant medical history. The computer retrieves the patient-related data from one or more databases, including the patient-supplied data inputs obtained during the check-in process, previous clinical notes, and any other patient-related data from the patient's medical records. An LLM prompt may instruct the LLM to prioritize or generate outputs based on the sources of patient data inputs. For instance, the LLM prompt instructs the LLM to generate text outputs having less-qualifying or more certain language for provider-supplied data, and more-qualifying or less certain language for patient-supplied data inputs.

In operation 203, the computer obtains subject-related data (e.g., patient data) for a subject interaction instance (e.g., patient visit) from one or more data sources. During a deployment phase, the computer uses the trained LLM to generate clinical visit notes based on clinical health data inputs. The trained LLM can be deployed to generate outputs representing strings of text that correspond to clinical visit notes, which can be integrated into the patient's medical records and used for downstream operations. The LLM can preprocess the data to standardize and normalize the data inputs.

The computer can access EHRs or other types of stored patient data to retrieve patient-related data, including medical history, previous diagnoses, treatment plans, and medication lists. The computer can also gather data from wearable devices and health monitoring systems, which provide real-time information on vital signs and other health metrics. The computer can integrate data from laboratory information systems, capturing test results and diagnostic reports relevant to the patient's visit. The computer can also obtain patient data from imaging systems, such as X-rays, MRIs, and CT scans, to include visual diagnostic information in the patient-related data. The computer can access patient-supplied data, such as symptoms and health concerns, entered at patient portals, mobile health applications, or patient intake forms using a patient computing device or kiosk.

In operation 205, the computer executes the trained components of the machine-learning architecture, such as a feature extractor, embedding extractor, or generative machine-learning model (e.g., LLM) to extract feature vectors from the subject-related data (e.g., patient data inputs). The computer can process structured data such as numerical values and categorical information from EHRs or other patient-related data. The computer can also handle unstructured data, such as clinical notes and patient-reported symptoms, by using NLP techniques to identify relevant medical terms and concepts. In some examples, the computer can convert these identified terms and concepts into feature vectors, which are numerical representations of features of the patient data. The LLM can use these feature vectors generate textual clinical notes. In another example, the computer can continuously update and refine the feature vectors as new patient data becomes available, such that that the generated clinical notes remain current with respect to updates in the patient's latest patient-related data.

The LLM is programmed, trained, and tuned to parse and process patient-related data and medical terminology in various inputs and generate the clinical notes, such as clinical notes for a patient visit or consultation with a provider. As mentioned, the LLM ingests the patient-related data, which includes structured data (e.g., medical or vital metric values, EHR data records), as well as unstructured data (e.g., patient-supplied intake information, transcriptions from discussion during patient visit, text of clinical notes). In some cases, the LLM may invoke the NLP operations to parse the various input data and identify relevant medical terms and concepts. In some cases, the LLM parses the input data in tokens, or tokenizes, the text into smaller units (e.g., words, subwords) and encode the tokens into the various feature vector or other form of numerical representations implemented by the LLM, which may represent medical terms or other patient-related information extracted from the patient data.

Generating the feature vectors includes executable operations of layers of the LLM or other machine-learning model of the machine-learning architecture, which execute transform functions to parse and transform raw text data into features and feature vectors as numerical representations processed by the LLM or other machine learning models. Embodiments described herein implement tokenization and one or more transformers (e.g., BERT, GPT) for extracting the feature vectors, but embodiments are not so limited. Non-limiting examples of the operations for generating the feature vectors include tokenization, transformers, word embeddings and vectorization, Term Frequency-Inverse Document Frequency (TF-IDF), Bag-of-Words (BoW), and Doc2Vec, and among others.

In operation 207, the computer executes the trained generative machine-learning model (e.g., LLM) to generate interaction data for the interaction instance based upon the subject-related data (e.g., generate a clinical note for the patient visit based on the patient data). Based on the feature vectors, the LLM is trained to identify patterns and relationships between medical terms that may be represented in the feature vectors and select the next tokens in order to generate the clinical note having a text description of the visit or patient information (e.g., health status, medical history, current visit details). For instance, the LLM structures the clinical note to include text for sections, such as patient history, examination findings, diagnosis, and treatment plan.

As mentioned, the LLM may be tune for specific medical terms and phrases to enhance the precision and clarity of the clinical note. This involves selecting the appropriate terminology based on the context provided by the patient data. The LLM may be further tuned on medical terminology datasets for structure of the text or clinical notes, such that the text of the clinical note adheres to medical documentation standards. The LLM continuously trains or tunes the weights or parameters to improve the text or clinical notes by cross-referencing the generated note against the original patient data. In this way, the computer continually trains or tunes the LLM at a given interval or in response to a condition, such as receiving new patient-related data from an EHR database or similar condition.

In some implementations, the LLM employs a sequence generation algorithm to predict the next token in the text based on the current context provided by the feature vectors. This involves using the learned patterns and relationships to determine the most appropriate medical terms and phrases to include in the clinical note. The LLM iteratively selects tokens, constructing sentences that includes text describing, for example, the patient health status, medical history, and current visit details. The LLM structures the clinical note to include text for sections such as patient history, examination findings, diagnosis, and treatment plan. For instance, when generating the patient history section, the LLM uses the feature vectors to identify relevant past medical events and conditions, ensuring that the narrative is comprehensive and accurate. As an example, for an examination findings section, the LLM selects tokens that describe the observed symptoms and clinical observations during the patient's visit. Throughout the generation process, the LLM continuously references the feature vectors to maintain coherence and relevance in the clinical note. The LLM is trained to predict the next token to maintain medical context and reflects the patient information.

In some cases, the computer feeds a selected customized LLM prompt and patient-related data to the LLM to generate the clinical note or narratives. The LLM prompt guides the LLM to generate the contextually relevant clinical note. For example, the LLM prompt may instruct the LLM to generate text containing a summary of the patient's current symptoms and any changes since the last visit, a description of relevant medical history and previous treatments, and a description of new patient data or observations made during the current visit, among other types of textual information.

In operation 209, the computer stores the interaction data (e.g., clinical note) into a database and may be referenced for various downstream software operations. The computer transmits the interaction data to a graphical user interface for display at a user device of a patient or service provider (e.g., service computing device 103a, patient computing device 106a).

### Discrepancy Detection

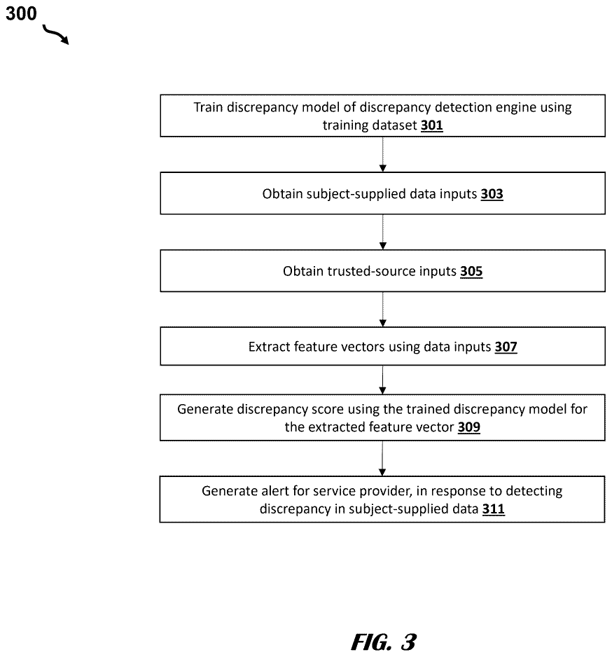

FIG. 3 shows a flowchart of operations of a computer-implemented method 300 for implementing machine-learning models of a machine-learning architecture for detecting discrepancies in patient-supplied data inputs, as compared against provider-supplied data or stored patient data. A computing device (e.g., analytics server 102, provider server 103b) executes software programming (e.g., discrepancy detection engine 130) for ingesting various types of patient-related data to identify or detect discrepancies between patient-supplied data inputs and trusted-source data inputs (e.g., provider-supplied data inputs, stored patient data inputs), which includes software routines for implementing machine-learning models of a machine-learning architecture to analyze various types of data inputs and identify or detect any such discrepancies. For ease of description and understanding, the operations of the computer-implemented method 300 are described as being performed by a computer, though embodiments may be implemented using any type of computing device and/or may be implemented using multiple computing devices.

In operation 301, the computer trains a machine-learning model (e.g., discrepancy model) of a discrepancy detection engine (e.g., discrepancy detection engine 130). During a training operational phase, the computer executes programming of the discrepancy detection engine to train the discrepancy model using corpora of a training dataset, including historical patient data and clinical notes from various data sources, such as electronic health records (EHRs), lab results, and patient-reported information. In an example, the computer can use supervised learning techniques to identify patterns and relationships within the patient-related data and training labels that indicate discrepancies. In an example, the training dataset can include labeled examples of consistent and inconsistent records, such that a loss function of the discrepancy model determines a level of error or loss and adjusts parameters or weights to identify the distinguishing features of the feature vectors. The computer determines that the detection model is trained when the level of error or loss satisfies a training threshold.

In operation 303, the computer obtains subject or patient-supplied data inputs, which include various forms of data or data sources received from the patient, patient intake forms, or patient devices. The discrepancy detection engine ingests the new patient-supplied data from various sources, through which the computer obtains patient-related data or information from the patient, such as patient-reported information entered at a patient form using a kiosk or patient device, patient-reported information of an audio transcript of a spoken conversation with a doctor, or other types of information originated from the patient (e.g., health metrics data from wearable devices).

In operation 305, the computer obtains trusted-source inputs (e.g., provider's service-supplied inputs and/or stored patient subject data) containing subject-related data. The discrepancy detection engine ingests the new trusted-source patient data from various sources, from which the computer obtains patient-related data as information from the provider or as stored data records retrieved from one or more databases. The stored patient data may include EHRs or other types of stored patient data, such as demographic information, lab results data, imaging data, and prior clinical notes, among other types of stored information about the patient. The provider-supplied data may include, for example, provider-reported information entered at provider computing device, provider-reported information of an audio transcript of a spoken conversation with the patient, or other types of information originated from the provider.

In operation 307, the computer executes layers of the discrepancy model (or other component of the discrepancy detection engine) to extract feature vectors. The feature vectors may include, for example, patient feature vectors extracted from patient-supplied data inputs and trusted feature vectors extracted using provider-supplied data inputs or stored patient data inputs. In an example, the discrepancy model can use NLP techniques to parse unstructured data or structured data in the data inputs and identify medical terms and concepts from the parsed data inputs to extract features representing terms or phrases of the parsed data. In this example, the discrepancy model can convert these terms and concepts into the feature vectors or other types of numerical representations of the patient data inputs. Non-limiting examples of extraction operations for generating the feature vectors may include transformers, TF-IDF, or word embeddings, among other operations for extracting the feature vectors.

In some embodiments, the computer executes preprocessing and normalizing operations on the patient-related input data for consistency and standardization. In such embodiments, the preprocessing operations can include normalizing numerical values, standardizing medical terminology, and anonymizing sensitive information.

In operation 309, the computer generates a discrepancy score using the trained discrepancy model for the extracted feature vectors. The discrepancy model determines the discrepancy score based on comparing the feature vectors. In this example, the trained discrepancy model can identify deviations from expected patterns by calculating the distance between the patient-supplied inputs and the trusted data inputs. The discrepancy score indicates a likelihood of discrepancies in the patient-supplied data inputs. In some implementations, the computer can use the feature vectors, which are numerical representations of the patient data inputs, to calculate distances between the feature vectors or features. The distance metric can be Euclidean distance, cosine similarity, or another appropriate measure that quantifies the difference between the feature vectors. As an example, a larger distance represented in the discrepancy score can indicate a higher likelihood of a discrepancy, while a smaller distance represented in the discrepancy score can indicate a lower likelihood of a discrepancy.

In some implementations, the discrepancy model can aggregate the calculated distances of certain features or feature vectors to generate an overall discrepancy score for the patient-supplied data. This score provides a quantitative measure of the potential inconsistencies within the patient-supplied data. The discrepancy engine may use the discrepancy score to prioritize the identified inconsistencies in certain features or feature vectors.

In operation 311, the discrepancy detection engine or other component of the computer may generate an alert for the provider, in response to the computer detecting a discrepancy in the patient-supplied data. For instance, the detected discrepancy model may detect and flag discrepancies based upon the extracted features or feature vectors, indicating discrepancies in the patient-supplied data, such as conflicting diagnoses, missing information, or contradictory treatment plans. The discrepancy detection engine may compare the discrepancy score(s) against one or more threshold values for discrepancy detection and detect a discrepancy when a discrepancy score satisfies a corresponding detection threshold.

In an example, the engine can create a summary report highlighting the inconsistencies and suggesting possible resolutions. In this example, the alerts can be integrated into the healthcare provider's workflow, allowing them to review and address the discrepancies promptly. In some examples, the engine can also provide recommendations for additional tests or follow-up actions to resolve the inconsistencies.

In operation 313, the discrepancy detection engine updates the patient records to reflect the resolved discrepancies. In an example, the engine can automatically correct minor inconsistencies based on predefined rules. In this example, significant discrepancies can be reviewed and approved by healthcare providers before updating the records. In some examples, the engine can also log the changes and maintain an audit trail for future reference.

### 3D Modeling Visualizations

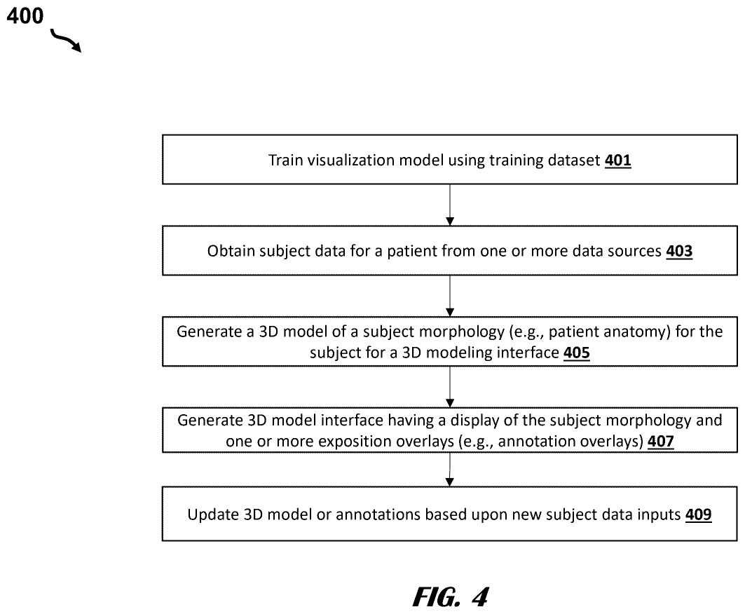

FIG. 4 shows a flowchart of operations of a computer-implemented method 400 for generating 3D anatomy models of patient anatomies for a 3D model interface. A computing device (e.g., analytics server 102, provider server 103b) executes software programming (e.g., visualization engine 126) for ingesting various types of patient-related data to generate the 3D models, which includes software routines for implementing machine-learning models of a machine-learning architecture to generate the 3D models. For ease of description and understanding, the operations of the computer-implemented method 400 are described as being performed by a computer, though embodiments may be implemented using any type of computing device and/or may be implemented using multiple computing devices.

In operation 401, the computer trains a machine-learning model (e.g., visualization model) of a visualization engine (e.g., visualization engine 126). The computer software routines for the visualization engine can train the visualization model using one or more corpora containing training patient data for any number of training patients or other training dataset, including training patient anatomy images, training labels indicating or mapping portions of anatomy images to medical terminology associated with the portions of the anatomy, and/or training annotations, among other types of training data. In some implementations, the training dataset can include various imaging data, such as MRI, CT scans, and X-rays. In some embodiments, the computer executes preprocessing operations. The computer may, for example, preprocess the images to normalize the data and enhance the quality of the images.

In operation 403, the computer obtains subject data for a subject from one or more data sources. For instance, the computer executes the trained machine-learning model during a deployment phase, on the various types of patient data received for a subject interaction instance (e.g., patient visit) or when a provider reviews and updates the patient data.

In operation 405, the computer generates a 3D model as visualized representation of a subject's morphology (e.g., patient anatomy) for the subject for a 3D modeling interface. For instance, the visualization engine can execute the trained visualization model to generate a 3D model of a patient anatomy. In an example, the model can reconstruct the anatomical structures in three dimensions, providing a detailed and accurate representation of the patient's anatomy. The computer may generate visual expositions (or visualized annotations) containing textual clinical notes and textual annotations, based on patient data, for display as overlays on the 3D model. In an example, the patient data can include medical records, lab results, and other relevant information. In this example, the visualization engine can use natural language processing techniques to extract relevant information from the patient data and generate annotations. In some examples, the annotations can be displayed as labels or markers on the 3D model, providing additional context and information to the user.

In operation 407, the computer generates the 3D model interface for display at a user computing device (e.g., provider computer, patient computer), where the 3D model interface includes 3D model of the patient anatomy and one or more expositions (e.g., visual annotations). For instance, the visualization engine executes software programming for rendering the 3D model and one or more overlays containing annotations related to portions of the patient anatomy. The 3D model interface may be interactive, such that a user (e.g., provider, patient) may interactively manipulate the 3D model or annotations to view different angles and sections of the anatomy or view further information about the portion of the anatomy or annotation.

In operation 409, the computer updates the 3D model or annotations based upon new patient data inputs. For instance, the visualization engine can continuously monitor the patient's medical records and update the 3D model and annotations as new information becomes available. The computer may receive inputs detect instances visualization engine may can ensure that the 3D model remains accurate and up-to-date, reflecting the latest patient data. In some examples, the visualization engine can notify the user of any significant changes or updates to the 3D model and annotations.

### Ambient Voice Capture

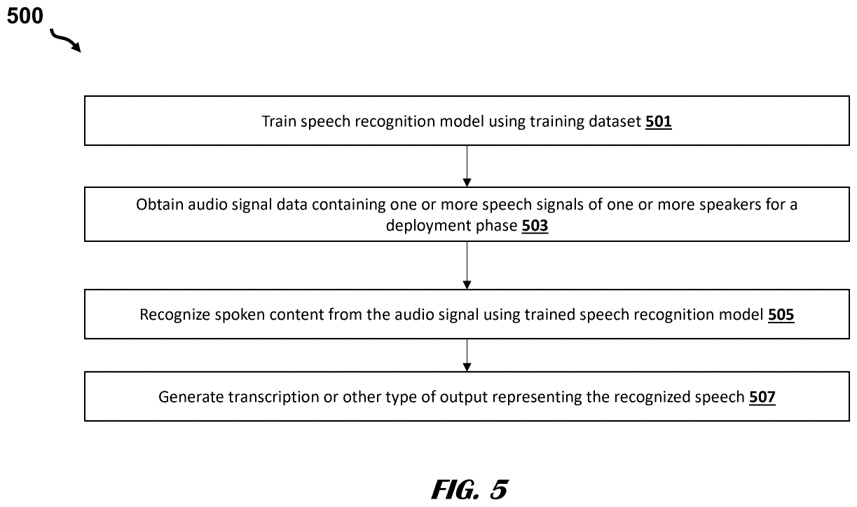

FIG. 5 shows a flowchart of operations of a computer-implemented method 500 for generating a text transcription or other form of output using audio signal data containing speech signals from one or more speakers (e.g., provider, patient). A computing device (e.g., analytics server 102, provider server 103b) executes software programming (e.g., speech recognition engine 124) for ingesting digital audio data to generate, identifying various types of patient-related data, and generating various outputs, such as clinical notes. As an example, a computer may execute software routines for implementing a speech recognition model of a machine-learning architecture programmed and trained to convert the digital audio data to the transcription output or clinical notes. For ease of description and understanding, the operations of the computer-implemented method 500 are described as being performed by a computer, though embodiments may be implemented using any type of computing device and/or may be implemented using multiple computing devices.

In operation 501, the computer trains one or more speech recognition models using a training dataset containing training audio signal or training audio data, among other types of data. During a training phase, the computer obtains (e.g., receives, retrieves) training audio data from various data sources for a plurality of training patients. The computer feeds the training dataset to the speech recognition engine to train the speech recognition model. The training data may include, for example, training audio signals or training audio data containing speech and, in some cases, corresponding transcriptions. The training dataset can include, for example, audio recordings containing audio data of audio signal captured from different environments and speakers, which the computer uses to train the speech recognition model for robustness.

In some embodiments, the computer executes operations to preprocess the audio signals or audio signal data. The computer can preprocess the audio signals to remove noise and enhance the quality of the recordings. The computer can preprocess the training audio signals to inject noise or otherwise reduce quality, such that the speech recognition model is trained for robustness using comparatively lower quality training audio data. The training dataset may further include training labels corresponding to the training audio data, indicating the expected outputs that should be generated by the speech recognition model.

The speech recognition model can be initialized with pre-trained weights or parameters, which can be fine-tuned using the collected training dataset. The computer feeds the training audio data into the speech recognition model and uses backpropagation to update the model's weights based upon comparing predicted outputs (generated by the speech recognition model) against expected outputs indicated by the training labels. A loss function compares the predicted outputs speech recognition model against the expected outputs (e.g., actual data or training labels) to determine a level of error or loss. In some cases, the speech recognition model includes a deep neural network (DNN) that learns to map audio features or feature vectors to, for example, corresponding phonemes and words. In this example, the DNN can be trained using supervised learning techniques, where the speech recognition model is provided with labeled training data containing the training audio signals and corresponding transcriptions or training labels. In some examples, the DNN includes a convolutional neural network (CNN) that extracts the various features from the audio signal data, or a recurrent neural network (RNN) that captures temporal dependencies in the speech data of the audio signal data, among other examples.

Optionally, the computer may extract a known-speaker embedding from known-speaker audio data. The computer may execute the speech recognition model using the known-speaker audio data (e.g., patient audio signal data, provider audio signal data) for a known-speaker (e.g., provider, patient), where the DNN extracts the features and feature vector corresponding to the known-speaker. The computer may then store this known-speaker embedding into memory for later reference during a speaker recognition operation. In an example, the computer can capture audio signal data containing speech from the patient or provider during an optional enrollment phase. The computer executes a feature extractor of the DNN may extract and generate a unique embedding that represents the known speaker's voice characteristics.

In operation 503, the computer obtains audio signal data containing one or more speech signals of one or more speakers for a deployment phase. The computer executing the speech recognition engine receives or receives the audio signal data containing speech of one or more speaker from one or more data sources, such as a microphone of the computer or a database containing stored audio signal data. In some cases, the computer can use microphones or other audio input devices to capture the audio signals. The captured audio signals can be digitized and stored in memory storage accessible to the computer for further processing. In some cases, the computer can continuously monitor the audio input to detect the presence of speech. In some cases, the computer can use voice activity detection to identify the segments of the audio signal that contain speech and discard the silent portions. The captured audio signals can then be processed by the speech recognition model to generate a transcription. In some embodiments, the computer can use speaker diarization techniques to distinguish between the speakers and assign the recognized speech to the correct individual.

As an example, a microphone placed in the examination room captures audio signals containing the speech dialogue between a patient and doctor. The computer obtains audio signal data containing the speech for the conversation between the doctor and the patient. As another example, the computer obtains audio signal data for a doctor's spoken dictation notes, where the doctor may use a handheld or wearable microphone to capture the doctor's voiced speech signal when dictating the spoken notes.

In operation 505, the computer recognizes spoken content from the audio signal data. The computer executes the speech recognition model trained to recognize the spoken content from the captured audio signal data. The computer can use the trained speech recognition model to analyze the audio signals and identify the spoken words or content. The speech recognition model is trained to convert the audio signal data into text by mapping the acoustic features of extracted feature vectors to corresponding phonemes and words. The computer extracts one or more current feature vectors for the current speakers in the current audio signal, which the trained speaker recognition model uses to identify the speech content and, in some cases, distinguish between the speakers.

As an example, the computer recognizes content in a conversation between a doctor and a patient. The computer can use the trained speech recognition model to process the audio signal data captured during the conversation. In this example, the trained speech recognition model can identify the spoken words of both the doctor and the patient, converting the speech into text. In some examples, the computer can use speaker diarization techniques to distinguish between the speakers and assign the recognized speech to the correct individual. The recognized text can then be used to generate a transcript of the conversation, which can be stored in the patient's medical records for future reference.

As another example, the computer recognizes content in a doctor's spoken dictation notes. The computer can use the trained speech recognition model to process the audio signals captured during the doctor's dictation in a provider microphone. In this example, the model can identify the spoken words of the doctor, converting the speech to textual data or other types of output.

In some implementations, the speech recognition engine may include the speech recognition model or other types of machine-learning models, such as a NLP model, trained to recognize medical terminology and associate the medical terms with specializations for contextualized detection and generate the text-based transcription. The speech recognition engine may format or otherwise generate the text of the transcription for a human-readable format or for input into a one or more downstream operations, such as a note generator engine or visualization engine. The speech recognition model may include one or more classifiers or other executable layers for speaker diarization, programmed or trained to distinguish between multiple speakers in the audio signal and assign the recognized portions of speech to particular speakers. In some cases, the speech recognition model may further identify the particular speaker identities and assign the recognized speech portions to the particular speakers. The computer can analyze the audio signal data to identify unique voice characteristics of the doctor and the patient. The computer may implement clustering algorithms to group audio segments of the audio signals data based on the feature vectors representing each speaker's voice characteristics, separating the speech of the doctor from that of the patient based upon a distance from the known-speaker feature vectors and a feature vector extracted for each particular current speaker.

Optionally, in some implementations, the computer executes operations for speaker recognition to identify and confirm identities of particular speakers in current audio signal data. The computer may, for example, determine or compute a recognition score for a current speaker based upon a distance or difference between a current-speaker feature vector extracted for a speaker in the current audio signal data against known-speaker embeddings stored in a database. The computer recognizes and identifies the speaker in the current audio signal as being a particular known-speaker in response to the computer determining that the recognition score generated for the speaker satisfies a speaker recognition threshold score or distance value.

In operation 507, the computer generates a transcription or other type of output representing the recognized speech. For instance, the speech recognition engine executing the speech recognition model can generate a transcription or other type of output representing the converted speech. In an example, the computer can format the recognized text into a readable transcription. In this example, the transcription can include punctuation and capitalization to enhance readability. In some examples, the computer can generate other types of outputs, such as subtitles for videos or commands for voice-controlled applications.

In an example of generating a transcription, the computer can convert the recognized speech from a doctor's spoken dictation notes into a formatted text document. In this example, the transcription can be structured with appropriate headings, paragraphs, and bullet points to organize the information clearly. In some examples, the computer can highlight key terms or phrases to make the transcription more useful for medical documentation. The recognized text can then be used to generate a transcript of the conversation. In an example, the computer can format the recognized text into a structured document, including timestamps and speaker labels to indicate who is speaking. In this example, the transcript can provide a clear and accurate record of the conversation, capturing all relevant details discussed between the doctor and the patient. In some examples, the computer can store the transcript in the patient's medical records for future reference, ensuring that the information is readily accessible for subsequent consultations and medical decision-making.

In another example of generating subtitles for videos, the computer can use the recognized speech from a recorded medical lecture to create subtitles. In this example, the computer can synchronize the subtitles with the video timeline, ensuring that the text appears at the correct times. In some examples, the computer can translate the recognized speech into different languages to provide multilingual subtitles for a broader audience.

In an example of generating commands for voice-controlled applications, the computer can use the recognized speech from a patient's interaction with a healthcare kiosk to execute specific actions. In this example, the computer can interpret the patient's spoken commands, such as “schedule an appointment” or “check my medical records,” and perform the corresponding actions. In some examples, the computer can provide voice feedback to confirm the actions taken, enhancing the user experience. The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, attributes, or memory contents. Information, arguments, attributes, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-Ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A computer-implemented method for generating subject-related information using machine-learning architectures of a data system, the method comprising:
obtaining, by a computer, subject-related data for a subject associated with a subject interaction instance from a plurality of data sources;
generating, by the computer, interaction data for the interaction instance using a generative machine-learning model based upon the subject-related data;
generating, by the computer, a 3D model image representing a morphology of the subject based on the subject-related data; and
generating, by the computer, an interactive 3D model user interface that includes the 3D model and an overlay of an exposition containing the interaction data associated with the morphology of the subject for display at a service computing device.

2. The method according to claim 1, further comprising receiving, by the computer, a service-supplied input from a service computing device interacting with the interactive 3D model user interface.

3. The method according to claim 2, further comprising updating, by the computer, a portion of the subject-related data for the subject based upon the service-supplied input entered to the interactive 3D model user interface.

4. The method according to claim 2, further comprising:
updating, by the computer, the 3D model and the exposition based on the subject-related data; and
updating, by the computer, the interactive 3D model user interface according to the 3D model and the exposition as updated for the service-supplied input.

5. The method according to claim 1, wherein obtaining the subject-related data includes:
obtaining, by the computer, an audio signal data containing a speech signal for one or more speakers including a service; and
generating, by the computer, a portion of the subject-related data based upon converting at least a portion of the speech signal of the service to text data for the portion of the subject-related data.

6. The method according to claim 5, wherein the computer receives the audio signal data via a microphone having the speech signal for the service and the subject during the interaction instance, and wherein the computer generates the interaction data using the subject-related data having the text data converted using the speech signal during the interaction instance.

7. The method according to claim 1, wherein generating the interaction data for the interaction instance using the generative machine-learning model includes obtaining, by the computer, a generative machine-learning model prompt stored in a database and containing instructs for the generative machine-learning model associated with the service for generating the interaction data.

8. The method according to claim 1, wherein obtaining the subject-related data includes:
extracting, by the computer, a first feature vector for subject-supplied input data using the subject-related data, and a second feature vector for trusted-source input data using the subject-related data; and
generating, by the computer, a discrepancy score for the subject-supplied input data based upon a distance between the first feature vector and the second feature vector.

9. The method according to claim 8, further comprising:
detecting, by the computer, a discrepancy in the subject-related data in response to determining that the discrepancy score satisfies a detecting threshold; and
generating, by the computer, an alert indicating the discrepancy for display at a user interface.

10. The method according to claim 1, wherein obtaining the subject-related data for the subject includes:
obtaining, by the computer, a subject-intake form from a database containing a plurality of templated forms; and
generating, by the computer, a user interface for displaying the subject-intake form at a subject device, wherein the computer receives at least a portion of the subject-related data as subject-supplied data inputs entered at the user interface having the subject-intake form.

11. A system for generating subject-related information using machine-learning architectures, the system comprising:
a server comprising at least one processor, configured to:
obtain subject-related data for a subject associated with a interaction instance from a plurality of data sources;
generate interaction data for the interaction instance using a generative machine-learning model based upon the subject-related data;
generate a 3D model image representing a morphology of the subject based on the subject-related data; and
generate an interactive 3D model user interface that includes the 3D model and an overlay of an exposition containing the interaction data associated with the morphology for display at a service computing device.

12. The system according to claim 11, wherein the server is further configured to receive a service-supplied input from a service computing device interacting with the interactive 3D model user interface.

13. The system according to claim 12, wherein the server is further configured to update a portion of the subject-related data for the subject based upon the service-supplied input entered to the interactive 3D model user interface.

14. The system according to claim 12, wherein the server is further configured to:
update the 3D model and the exposition based on the subject-related data; and
update the interactive 3D model user interface according to the 3D model and the exposition as updated for the service-supplied input.

15. The system according to claim 11, wherein when obtaining the subject-related data the server is configured to:
obtain an audio signal data containing a speech signal for one or more speakers including a service; and
generate a portion of the subject-related data based upon converting at least a portion of the speech signal of the service to text data for the portion of the subject-related data.

16. The system according to claim 15, wherein the server receives the audio signal data via a microphone having the speech signal for the service and the subject during the interaction instance, and wherein the server generates the interaction data using the subject-related data having the text data converted using the speech signal during the interaction instance.

17. The system according to claim 11, wherein when generating the interaction data for the interaction instance using the generative machine-learning model the server is configured to obtain a generative machine-learning model prompt stored in a database and containing instructs for the generative machine-learning model associated with the service for generating the interaction data.

18. The system according to claim 11, wherein when obtaining the subject-related data, the server is configured to:
extract a first feature vector for subject-supplied input data using the subject-related data, and a second feature vector for trusted-source input data using the subject-related data; and
generate a discrepancy score for the subject-supplied input data based upon a distance between the first feature vector and the second feature vector.

19. The system according to claim 18, wherein the server is further configured to:
detect a discrepancy in the subject-related data in response to determining that the discrepancy score satisfies a detecting threshold; and
generate an alert indicating the discrepancy for display at a user interface.

20. The system according to claim 11, wherein when obtaining the subject-related data for the subject the server is further configured to:
obtain a subject-intake form from a database containing a plurality of templated forms; and
generate a user interface for displaying the subject-intake form at a subject device, wherein the server receives at least a portion of the subject-related data as subject-supplied data inputs entered at the user interface having the subject-intake form.
